(19) United States
(12) Patent Application Publication
(10) Pub. No.: US 2024/0408341 A1
(43) Pub. Date: Dec. 12, 2024

(54) HEADGEAR FOR A RESPIRATORY INTERFACE

(71) Applicant: Fisher & Paykel Healthcare Limited, Auckland (NZ)

(72) Inventors: Jake Baker HOCKING, Auckland (NZ); Bruce Michael WALLS, Auckland (NZ); Katie O'BRIEN, Auckland (NZ)

(21) Appl. No.: 18/262,720

(22) PCT Filed: Feb. 3, 2022
(86) PCT No.: PCT/NZ2022/050013

§ 371 Date: Jul. 24, 2023

Related U.S. Application Data
(60) Provisional application No. 61/200,012, filed on Nov. 24, 2008.

(51) Int. Cl. A61M 16/06

(52) U.S. Cl. CPC A61M 16/0683; A61M 16/0605

(57) ABSTRACT

Headgear that secures a respiratory interface to the head of a patient. The respiratory interface may be used to treat obstructive respiration diseases or sleep apnoea. The headgear comprises a positioning web having a height and a length that extends at least partly about the head of a user when the headgear is fitted to the user. The positioning web includes at least one pathway for at least one tension line. Furthermore, the positioning web maintains the positioning of the one or more pathways about the head of the user and the positioning web is expandable and contractible between an operative configuration when fitted to the user and an expanded configuration to facilitate donning and doffing of the headgear respectively.

### FIELD OF THE INVENTION

The present invention relates to treatments for obstructive respiration diseases and for sleep apnoea. In particular, the invention relates to headgear that secures a respiratory interface to the head of a patient.

### BACKGROUND

Respiratory interfaces are used in a range of different treatments for sleep apnea and for respiration diseases, including CPAP (continuous positive airway pressure), NIV (non-invasive ventilation) and high-flow. With these different treatments, there are different interfaces, but two common features exist among them.

A function of the headgear is to restrain the interface in response to forces typically encountered in-use, such as blow-off forces and hose-pull forces, to keep the interface in position against the face of the user. For therapies such as CPAP and NIV the headgear is also generally required to maintain the interface in the form of a mask in a sealing arrangement with the face of the user.

Existing headgear involves strapping an interface to the face, but people tend to overtighten the headgear, in particular to reduce gas leakage, and with the effect that too much force is applied. This results in discomfort and a reduction in compliance. There is an on-going challenge to improve patient comfort whilst providing adequate forces to hold the interface in place, and more specifically for masks to adequately seal with the user. Improving the interface stability and comfort will enable patients to sleep in their preferred position, including on their side, back, and front, with reduced interface displacement, which can cause gas leakage from the interface. If leaks occur, patients don't receive the optimum treatment and can experience respiratory gas being blown in their eyes which causes discomfort.

As such, there is an ongoing challenge to provide a better balance between seal stability and patient comfort.

### SUMMARY OF THE DISCLOSURE

In one aspect, there is provided headgear for a respiratory interface comprising a positioning web having a height and a length that extends at least partly about the head of a user when the headgear is fitted to the user and the positioning web includes:
at least one pathway for at least one tension line, the at least one pathway comprising;
   a first pathway portion extending along at least part of the length of the positioning web, and
   a second pathway portion extending along at least part of the length of the positioning web, wherein
the first and second pathway portions are spaced apart by a varying distance as they extend along the positioning web;
and wherein the positioning web:
   maintains the positioning of the one or more pathways about the head of the user; and
   is expandable and contractible between an operative configuration when fitted to the user and an expanded configuration to facilitate donning and doffing of the headgear respectively.

References to the height and the height direction refer to the direction between the top of the user's neck and the top of a user's head. In addition, references to the length and length direction refer to the direction generally horizontally about a user's head. In embodiments where the positioning web is a loop, the length-direction refers to the circumferential or peripheral direction about the loop.

The positioning web may maintain the pathways portions in spaced relationship along the length of the positioning web.

The spaced relationship between the pathway portions may vary over at least a portion of the length of the positioning web.

The positioning web may maintain the pathway portions in parallel relationship along at least a portion of the length of the positioning web.

The first and second pathway portions may converge along the length of the positioning web.

The first and second pathway portions may converge and diverge along the length of the positioning web.

The first pathway portion and the second pathway portion may be portions of a single pathway.

The positioning web may comprise first and second pathways, wherein the first pathway comprises the first pathway portion and the second pathway comprises the second pathway portion.

The first and second pathway portions may intersect.

The first pathway portion and the second pathway portion may intersect at least once at a point where the distance between the first pathway portion and the second pathway portion is zero.

The first and second pathway portions may intersect at an intersection region of the positioning web.

The intersection region may be located at a rear part of the positioning web. The intersection region may be located at a central rear part of the positioning web. In some embodiments, the intersection region may be located at a front part of the positioning web. The intersection region may be located at a central front part of the positioning web. In some embodiments, the intersection region may be above the level of the user's ear. For instance, the intersection may be at the top of the user's head.

The front part of the positioning web may be adapted to extend across the respiratory interface in use.

The first and second pathway portions may intersect twice in two distinct intersection regions of the positioning web.

The two intersection regions may coincide respectively with front and rear parts of the positioning web. The two intersection regions may coincide respectively with central front and central rear parts of the positioning web.

The two intersection regions may coincide with respective side parts of the positioning web.

The positioning web may comprise a first region at a front lower part of the positioning web, a second region at a rear lower part, a third region at a rear upper part above the second region and a fourth region at a front upper part above the first region.

The first and second regions may meet on a first side in front of or over or behind the ears of a user when the headgear is fitted.

The first and second regions may meet on a second side in front of or over or behind the ears of a user when the headgear is fitted.

The fourth and third regions may meet on the first side in front of or over or behind the ears of a user when the headgear is fitted.

The fourth and third regions may meet on the second side in front of or over or behind the ears of a user when the headgear is fitted.

The first and second pathway portions may lie adjacent one another in one or more of the first, second, third and fourth regions of the positioning web.

The positioning web may have a fifth region that connects the first side of the positioning web to the second side of the positioning web and that is configured to extend across a top of the user's head when the headgear is fitted.

The fifth region may include the first pathway portion, the second pathway portion or the first and second pathway portions to enable one or more tension lines to extend between the first and second sides of the positioning web.

The first and second pathway portions are merged in at least part of the fifth region.

The fifth region may be configured to connect to the first side and the second side adjacent to the ears of the user when fitted.

The height of the positioning web may comprise the spacing of the pathways plus the dimension of the pathways in the width direction.

The one or more pathways for the one or more tension lines may be a channel or tube.

The one or more pathways may enclose a portion of respective one or more tension lines.

The one or more pathways may extend along an external side of the first, second, third, fourth and/or fifth regions of the positioning web.

The positioning web may be non-elastically expandable. For example, the positioning web may comprise telescoping portions that are capable of moving to extend the positioning web in its length direction.

The positioning web may be elastic.

The positioning web may locate the at least one pathway in an operating position about the user's head.

The elasticity of the positioning web may be anisotropic.

One or more regions of the positioning web may have an elasticity that is different to the elasticity of other regions. The difference in elasticity may be provided by differences in construction between the regions, differences in materials in the regions or a combination of both.

The elasticity of the positioning web may be different in the height-direction of the positioning web compared to the elasticity in the length-direction of the positioning web.

The elasticity of the positioning web may be greater in the length-direction than the elasticity in the height-direction.

The elasticity of the positioning web may be different in a lateral direction compared to the elasticity in a circumferential direction.

The elasticity of the positioning web in a lateral direction may be greater than the elasticity in the circumferential direction.

When the positioning web is in the expanded configuration, the positioning web may be relatively energised in the length-direction compared to when the positioning web is in the operative configuration.

When the positioning web is in the expanded configuration, the positioning web may be relatively energised in the length-direction compared to the height-direction. This is, when in the expanded configuration, the positioning web may have more stored energy in the length-direction than in the height direction, and thereby has greater potential to contract in the length-direction than the height direction.

When the positioning web is in the operative configuration, the positioning web may be relatively relaxed in the length-direction compared to when in the expanded configuration.

When in the expanded configuration, the positioning web may have substantially equal potential energy in the length-direction and the height-direction.

The positioning web may have a greater resistance to folding or buckling in the height-direction than in the length-direction.

In other words, the positioning web may have greater rigidity in the height-direction than in the length-direction. This can assist the positioning web in maintaining the positioning of the one or more pathways about the head of the user in the height-direction.

In one example, the positioning web may be self-supporting in the height-direction.

The positioning web may be the same dimension in the height-direction for when the positioning web is in the expanded and operative configurations.

The positioning web may have a structure that permits expansion and contraction of the positioning web or the headgear. This is termed as mechanically expandable and contractible.

The mechanical expansion and contraction may be in the length direction, may be in the height direction or may be in both the length direction and the height direction.

The mechanically expandable and contractible positioning web may be formed of elastic material or substantially inelastic material or may be formed of material with anisotropic elasticity.

The positioning web may include substantially inelastic portions that move relative to each other so that the positioning web is mechanically expandable and contractible.

For instance, the positioning web may have telescopically extending portions or portions on rails that slide over each other to expand and contract the positioning web. In another instance, the positioning web may comprise two or more overlapping sections which are translatable relative to each other to permit expansion and contraction of the positioning web. The overlapping sections may be separated by folds in the positioning web.

The headgear may include inelastic portions, such as panels, that are joined together by elastic portions which allow the positioning web to expand and contract.

The positioning web may be elastic in the height direction and may be relatively inelastic, but mechanically expandable and contractible, in the length direction.

The positioning web may be elastic in the length direction and may be relatively inelastic, but mechanically expandable and contractible, in the height direction.

The inelastic portions may include scales (or sheets or tiles) that at least partially slide over each other. In one example, the scales may be arranged to slide over each other linearly in a telescoping manner. In another example, the scales may be arranged to slide over each other in a multi-directions (dimensions) to form a blanket (or panel) that can contract and expand. The scales may have a curvature to facilitate sliding of the scales on each other. The scales may be sized and shaped to allow the positioning web to conform to the shape of the user's head.

For example, the scales may have less curvature for regions of the headgear at, or adjacent to, a temporal region of a user's head. In another example, the scales may have more curvature for regions of the headgear at, or adjacent to the rear of the user's head.

Alternatively, the position web may comprise relatively inelastic portions and mechanically expandable and contractible portions. For example, the one or more pathways or pathway portions may be relatively inelastic and may be connected to discrete, spaced apart panels which are mechanically expandable and contractible.

The positioning web may include elastic portions, such as regions with anisotropic elasticity, or regions with substantially consistent elasticity, so that the positioning web is mechanically expandable and contractible.

The positioning web may be formed as a closed loop.

The positioning web may be co-operable with a respiratory interface to form a closed loop.

The headgear may further comprise one or more tension lines. For instance, the headgear may comprise one tension line for each pathway.

The or each tension line may be operable to hold the positioning web in the operative configuration when the headgear has been donned.

The or each tension line may prevent the positioning web from inadvertently expanding from the operative configuration. For example, the or each tension line may prevent accidental movement of the headgear by blow-off or tube-pull forces.

The or each tension line are arranged to enable an external force acting on the headgear to be transferred to the tension lines which extend in different pathways and in turn distribute the external force about the head of the patient, and thereby reduce movement of the headgear relative to the patient's head.

By the positioning web maintaining the spacing of the pathway portions, the spacing of the or each tension line is maintained.

In the event of an external force acting on the headgear, the tension in the or each tension line can counteract the external force.

The or each tension line may extend from the first side of the positioning web to the second side of the positioning web, and when the headgear is subject to a pulling force, the tension in the or each tension line increases in the respective first side or the second side, and can also increase in the opposite first side or second side, thereby maintaining the headgear in position on the patient.

The or each tension line may vary in position in the height-direction from the first side and the second side of the headgear and may crossover at an intersection, thereby tension in the tension line can be transferred from the first side to the second side and vice versa.

The position of the tension line in the height-direction may differ from the first side of the positioning web to the second side of the positioning web. For instance, one tension line may be at a lower position in the height-direction on the first side and at an upper position in the height-direction on the second side, and the same or another tension line may be at an upper position in the height-direction on the first side and at a lower position in the height-direction on the second side, in which the one tension line intersects itself or both tension lines intersect at the intersection point.

When the headgear is subject to a pulling force that is either upward or downward, tension in the tension lines can respond by either increasing or decreasing to maintain the position of the headgear on the patient.

For example, in the event of the pulling force being upward or downward, tension in a lower tension lines on the first side and/or the second side can increase and the tension on an upper tension lines on the first side and/or second sides can increase, thereby distributing the pulling force about the head of the patient via the headgear and maintaining the positioning web of the headgear in the operative configuration.

The tension line may comprise a first tension line portion and a second tension line portion and wherein the first tension line portion co-operates with the first pathway portion to extend along the positioning web, and the second tension line portion co-operates with the second pathway portion to extend along the positioning web.

The one or more tension lines are less capable of expanding and contracting compared to the expandability and contractibility of the positioning web.

The one or more tension lines may be less elastic than the positioning web.

The one or more tension lines may limit the maximum expandability of the positioning web.

The maximum expandability of the positioning web may be limited to the deployed length of the tension line.

The one or more tension lines may be substantially inelastic.

The one or more tension lines may have some elasticity.

The one or more tension lines may have an elastic modulus that is at least 120% or 150% or 200% or 300% or 400% of the elastic modulus of the positioning web.

The one or more tension lines may exhibit an elastic strain that is less than 5% or 4% or 3% or 2% or 1% at tensions associated with blow-off and tube-pull forces.

The one or more tension lines may have an elastic strain that varies along the length of the or each tension line for a given tension applied to the tension line.

The one or more tension lines may comprise a plurality of sections and the elastic strain of at least one section differs from the elastic strain of another section for a given tension applied to the tension line.

The elastic strain of the section or sections forward of the user's ears may be less than the elastic strain of the section or sections rearward of the user's ears.

Sections with a higher elastic strain than the elastic strain of other sections may exhibit elastic strain greater than 5% while the tension line exhibits an overall elastic strain that is less than 5%. The overall elastic strain may be less than 5% at tensions associated with blow-off and tube-pull forces.

The one or more tension lines may have an elastic modulus that varies along the length of the or each tension line.

The one or more tension lines may comprise a plurality of sections and the elastic modulus of at least one section differs from the elastic modulus of another section.

The elastic modulus of the section or sections forward of the user's ears may be greater than the elastic modulus of the section or sections rearward of the user's ears.

Sections with a lower elastic modulus than the elastic modulus of other sections may exhibit elastic strain greater than 5% while the tension line exhibits an overall elastic strain that is less than 5%. The overall elastic strain may be less than 5% at tensions associated with blow-off and tube-pull forces.

The one or more tension lines may have a composition that varies along the length of the tension line. The one or more tension lines may have a composition forward of the user's ears that has a higher elastic modulus than an elastic modulus of a composition of the tension line rearward of the user's ears.

The one or more tension lines may have a cross-sectional area that differs along the length of the tension line. At least one section has a cross-sectional area that differs from the cross-sectional area of other section.

The one or more tension lines may comprise an assembly of different materials. The sections of the one or more tension lines may comprise different materials.

The one or more tension lines may exhibit a strain that is less than 5% or 4% or 3% or 2% or 1% at tensions of up to 50N, preferably up to 40N, preferably up to 30N, more preferably up to 20N.

The one or more tension lines may be sufficiently elastic to stretch to account for small changes in tension. These small changes in tension might occur, for example, due to the user adjusting their head position during sleep.

The one or more tension lines may be substantially inelastic at tensions associated with blow-off and tube-pull forces. For example, the tension line may be inelastic compared to the expandability and contractibility of the positioning web for tensions of at least double the blow-off and tube-pull forces.

The one or more tension lines may be substantially inelastic under a tensile force of up to 50N, preferably up to 40N, preferably up to 30N, more preferably up to 20N.

The relative sliding of the pathway and the tension line operates to reduce a tension differential in the tension line in different parts of the pathway. Ideally, the tension line slides freely in the pathway such that there is uniform tension in the tension line throughout the pathway.

The one or more tension lines may be slidable relative to the or each pathway to enable extension and/or contraction of the positioning web.

The headgear may further comprise a tension-line length controller which is operable to control the length of tension line deployed to the or each pathway.

The controller may enable selective switching between the operative configuration and the donning and/or doffing configuration.

The controller may assist in the contraction of the positioning web by decreasing the length of tension line deployed in the pathway.

The controller may be operable to control fit of the headgear in the operative configuration by adjustment of the length of tension line deployed to the or each pathway.

The controller may include an accumulator which stores tension line that is not deployed to the or each pathway.

The controller may include a lock which fixes the deployed length of tension line.

The lock may disable operation of the controller to fix the deployed length of the tension line.

In some embodiments, the controller and the accumulator may be combined and comprise a reel about which tension line can be wound to store tension line, and wherein winding of the reel controls deployment of the tension line.

The reel may be disposed within a housing.

In some embodiments, the combined controller and accumulator may include a dial that is rotationally linked to the reel such that rotation of the dial causes winding of the reel.

The lock may be provided by formations on the dial and the housing which are engageable to prevent rotation of the dial and are disengageable to permit rotation of the dial.

The controller may be disposed at a location that is near end points of the pathway portions so that the one or more tension lines feed from the controller into the pathway portions.

The controller may be disposed at a location that is near end points of the first and second pathway portions so that the one or more tension lines feed from the controller into the first and second pathway portions.

The controller may be disposed on the third region of the web so that the one or more tension lines feed from the controller into the third and/or fourth pathway portions.

The headgear may further include a patient interface and one or more tension-line length controllers which link the one or more tension lines to the patient interface to transfer tension to the interface and which controller is operable to selectively disconnect and reconnect the one or more tension lines to the patient interface.

The or each tension-line length controller may comprise a friction resistor which interacts with the or each tension line to permit adjustment of the deployed length of tension line when tension in the tension line overcomes friction applied by the friction resistor.

In some embodiments, the friction resistor may be a spool of the reel about which the one or more tension lines are wound.

One end of each one or more tension line may be fixed to the patient interface and the other end of each tension line feeds into the friction resistor so that the deployed length of the or each tension line is adjustable.

In some embodiments, the friction resistor is coupled with the lock and wherein length adjustment of the deployed length of tension line is enabled by unlocking the lock and is disabled by locking the lock.

In an alternative, one end of each tension line is releasably linked to the patient interface by the controller so that a deployed length of each tension line can be set by adjustment of the controller and the deployed length is retained upon unlinking of the tension line from the patient interface, whereby the headgear can be re-fitted by re-linking the tension lines, having the previously set deployed length, to the patient interface.

An end of the at least one pathway may be at the front of the positioning web.

An end of the at least one pathway may be at or adjacent to a location associated with a respiratory interface.

An end of the at least one pathway may be at a central front part of the positioning web.

An end of the at least one pathway may be at the side of the positioning web.

An end of the at least one pathway may be at a location on a side of the positioning web that is adjacent the third region.

An end of the at least one pathway may be within the third region of the positioning web.

An end of the at least one pathway may be within the third region at or adjacent to a point that is midway between the first and second sides of the positioning web.

Each pathway ends at a location where the respective tension line transitions from being within the pathway to being outside the pathway.

The first pathway portion may be positioned above the second pathway portion on a first side of the user and the first pathway portion may be positioned below the second pathway portion on a second side of the user opposite to the first side, when the positioning web is fitted to the user.

In one embodiment, the first pathway portion may be positioned above the ear of the user on a first side of the user and positioned below the ear of the user on a second side of the user opposite to the first side and the second pathway portion may be positioned above the ear of the user on the second side of the user and positioned below the ear of the user on the first side, when the positioning web is fitted to the user.

The first side may be a right side of the positioning web and the second side may be a left side of the positioning web.

The first pathway portion may extend along the first side in the first and second regions and then extends along the second side in the third and fourth regions and the second pathway portion may extend along the second side in the first and second regions and then extends along the first side in the third and fourth regions.

The first and second pathway portions may be portions of a single pathway that is continuous through the fourth region.

Ends of the first and second pathway portions may be located in a respiratory interface region.

In one embodiment of the headgear, the first pathway portion may be positioned above the ear of the user on a first side of the user and positioned below the ear of the user on a second side of the user opposite to the first side, when the positioning web is fitted to the user.

The first side may be a left side of the positioning web and the second side may be a right side of the positioning web.

The first pathway portion may extend along the first side in the fourth and third regions and then extends along the second side in the second and first regions and the second pathway portion may extend along the second side in the fourth and third regions and then extends along the first side in the second and first regions.

The first and second pathway portions may be portions of a single pathway that is continuous through the first region.

Ends of the first and second pathway portions may be located in a respiratory interface region.

The first pathway portion and the second pathway portion may be separate pathways.

Tension lines disposed in each of the first and second pathways include a break-fit connection which releases tension in the tension line when a threshold tension is applied to the tension line without changing the deployed length of the tension line.

In one embodiment of the headgear:
(a) the first pathway portion may commence at the fifth region of the positioning web and extends forwardly of the fifth region in the fourth region on the first side, then transitions into the first region and extends rearwardly in the first and second regions on the second side, then transitions into the third region and extends along the first side back to the fifth region; and
(b) the second pathway portion may commence at the fifth region of the positioning web and extends forwardly of the fifth region in the fourth region on the second side, then transitions into the first region and extends rearwardly in the first and second regions on the first side, then transitions into the third region and extends along the second side back to the fifth region.

The first and second pathway portions may intersect forward and rearward of the fifth region.

In another embodiment of the headgear:
(a) the first pathway portion may commence at the fifth region on the first side of the positioning web, transition into the fourth region and then the first region forwardly of the fifth region, then extend rearward on the first side along the first and second regions, then transition into the third region and extend into the fifth region on the second side; and
(b) the second pathway portion may commence at the fifth region on the second side, transition into the fourth region and then the first region forwardly of the fifth region, then extend rearward on the second side along the first and second regions, then transition into the third region and extend into the fifth region on the first side.

The headgear may include a respiratory interface region and the first and second pathway portions transitions from the fourth region to the first region via or adjacent to the respiratory interface region.

In another embodiment of the headgear:
(a) the first pathway portion may commence at the fifth region on the first side of the positioning web, transition into the fourth region and then the first region forwardly of the fifth region, then extend rearward on the second side along the first and second regions, then transition into the third region and extend into the fifth region on the second side; and
(b) the second pathway portion may commence at the fifth region on the second side, transition into the fourth region and then the first region forwardly of the fifth region, then extend rearward on the first side along the first and second regions, then transition into the third region and extend into the fifth region on the first side.

The headgear may include a respiratory interface region and the first and second pathway portions transition from the fourth region to the first region via or adjacent to the respiratory interface region.

The first and second pathway portions transition between the second and third regions rearward of the fifth region.

The first and second pathway portions transition between the second and third regions at a rear part of the positioning web.

In another embodiment, the first pathway portion may extend from the third region on the first side, to the fifth region on the second side, then across the fifth region to the fourth region on the first side and forwardly along the fourth region and the second pathway portion extends from the third region on the second side, to the fifth region on the first side, then across the fifth region to the fourth region on the second side and forwardly along the fourth region.

The first and second pathway portions commence at a location adjacent the fifth region on the first side and on the second side respectively.

The first pathway portion may extend from the third region to the second region on the first side and then from the second region to the third region on the second side before crossing the fifth region.

The second pathway portion may extend from the third region to the second region on the second side and then from the second region to the third region on the first side before crossing the fifth region.

The first pathway portion and the second pathway portion are portions of a single pathway which is continuous across the fourth region.

The first and second pathway portions may transition into the first region from the fourth region.

In this embodiment, the first pathway portion may extend rearwardly along the first side from the fourth region to the first region, then to the second region, the first pathway portion then extends into the third region and then extends forwardly along the first side into the fourth region and then the first region on the first side and the second pathway portion may extend rearwardly along the second side from the fourth region to the first region, then to the second region, the first pathway portion then extends into the third region and then extends forwardly along the second side into the fourth region and then the first region on the second side.

In a variation of this embodiment, the first pathway portion may extend rearwardly along the first side from the first region to the fourth region, then to the third region, the first pathway portion then extends into the second region and then extends forwardly along the first side into the first region and then the fourth region on the first side and the second pathway portion may extend rearwardly along the second side from the first region to the fourth region, then to the third region, the first pathway portion then extends into the second region and then extends forwardly along the second side into the first region and then the fourth region on the second side.

In this embodiment and the variation, the first and fourth regions respectively are adapted to meet the second and third regions on the first and second sides over or behind the ears of the user when the headgear is fitted.

In this embodiment and the variation, the first pathway portion and the second pathway portion may be portions of a single pathway which crosses the respiratory interface region.

Ends of the first and second pathway portions may be located in a respiratory interface region.

In another embodiment, the first pathway portion may extend rearwardly along the first region on the first side, into the second region on the first side, into the third region at the rear of the positioning web, then along the fifth region from the second side to the first side and forwardly along the fourth region and the second pathway portion may extend rearwardly along the first region on the second side, into the second region on the second side, into the third region at the rear of the positioning web, then along the fifth region from the second side to the first side and forwardly along the fourth region.

The first pathway portion and the second pathway portion may be portions of a single pathway which crosses the respiratory interface region.

The first pathway portion and the second pathway portion may be portions of a single pathway which is continuous across the fourth region.

Ends of the first and second pathway portions may be located in a respiratory interface region.

In a variation of this embodiment, the first pathway portion may extend rearwardly along the fourth region on the first side, then along the fifth region from the first side to the second side, into the third region on the second side, into the second region at the rear of the positioning web, and forwardly along the first region on the first side and the second pathway portion may extend rearwardly along the fourth region on the second side, then along the fifth region from the second side to the first side, into the third region on the first side, into the second region at the rear of the positioning web, and forwardly along the first region on the second side.

The first pathway portion and the second pathway portion may be portions of a single pathway which crosses the respiratory interface region.

The first pathway portion and the second pathway portion may be portions of a single pathway which is continuous across the first region.

Ends of the first and second pathway portions may be located in a respiratory interface region.

The first and second pathway portions may intersect. The intersection may be at the rear of the headgear.

The first and second pathway portions may intersect twice. The intersections may be at the rear of the headgear and in the fifth region.

In another embodiment, the first pathway portion may extend rearwardly along the first region along the first side, into the fourth region, then the third region and then along the second region from the first side to the second side, the first pathway portion then extends into the third region, then along the fifth region from the second side to the first side and then forwardly along the fourth region on the first side and the second pathway portion may extend rearwardly along the first region along the second side, into the fourth region, then the third region and then along the second region from the second side to the first side, the second pathway portion then extends into the third region, then along the fifth region from the first side to the second side and then forwardly along the fourth region on the second side.

The first pathway portion and the second pathway portion may be portions of a single pathway which crosses the respiratory interface region.

The first pathway portion and the second pathway portion may be portions of a single pathway which is continuous across the fourth region.

In another embodiment, the first pathway portion may extend rearwardly along the fourth region on the first side, along the third region from the first side to the second side and then forwardly along the fourth region on the second side and the second pathway portion may extend rearwardly along the fourth region, then along the fifth region from the first side to the second side and then forwardly along the fourth region on the second side.

In another embodiment, the first pathway portion may transition from the third region to the second region on the first side, extend along the second region about the rear of the positioning web and from the second region to the third region on the second side.

In another embodiment, the first pathway portion may extend from the fifth region on the first side, then around the fourth region from the first side to the second side and then into the fifth region on the second side and the second pathway portion may extend from the fifth region to the third region on the first side and then along the third region from the first side to the second side and into the fifth region on the second side.

The first pathway portion may transition from the fourth region to the first region on the first side and then back to the fourth region on the second side.

The second pathway portion may transition from the third region to the second region on the first side and then back to the third region on the second side.

In another embodiment, the first pathway portion may extend rearwardly along the first region on the first side and then across the fifth region to the second side and forwardly along the first region and the second pathway portion may extend rearwardly along the first region on the first side, along the third region about the rear of the positioning web and then forwardly along the fourth region on the second side.

The first pathway portion may transition between the first region and the fifth region via the fourth region, the third region or the second region and either the third region or the fourth region.

In another embodiment, the first pathway portion may extend rearwardly along the first region on the first side, then along the second region from the first side to the second side and then forwardly along first region on the second side and the second pathway portion may extend from the fifth region to the fourth region on the first side, along the fourth region from the first side to the second side and then into the fifth region on the second side.

In another embodiment, the first pathway portion may extend from the fifth region on the first side to the fourth region, forwardly along the fourth region and then extend rearwardly along the first region on the first side and the second pathway portion may extend from the fifth region on the second side to the fourth region, forwardly along the fourth region and then extend rearwardly along the first region on the second side.

The transitions of the first and second pathway portions from the fourth region to the first region may be forward of the third region.

The transitions may be at the respiratory interface region.

The first and second pathway portions may commence in the fifth region and terminate in the second region respectively on the first side and on the second side.

The first and second pathway portions may terminate adjacent or rearward of the fifth region.

In another embodiment, the first pathway portion may extend rearwardly along the fourth region on the first side and then forwardly along the first region on the first side and the second pathway portion may extend rearwardly along the fourth region on the second side and then forwardly along the first region on the second side.

The first and second pathway portions may transition respectively from the first region to the fourth region via the second and/or third regions on the respective first and second sides.

The first and second pathway portions may be adapted to transition respectively from the second region to the first region and from the first region to the second region rearward of the ears of the user when the headgear is fitted.

Ends of the first and second pathway portions may be located in or adjacent to the respiratory interface region.

In another embodiment, the first pathway portion may extend rearwardly along the fourth region on the first side and then forwardly along the first region on the first side and the second pathway portion may extend rearwardly along the fourth region on the second side and then forwardly along the first region on the second side.

The first and second pathway portions may be adapted to transition from the first region to the fourth region forward of the ears of the user when the headgear is fitted.

In another embodiment, the first pathway portion may extend from the first region to the fourth region at or adjacent to a respiratory interface region and then extend rearwardly along the fourth region on the first side, then forwardly along the first region on the first side and the second pathway portion may extend from the first region to the fourth region at or adjacent to a respiratory interface region on the second side and then extend rearwardly along the fourth region on the second side and then forwardly along the first region on the second side.

The first and second pathway portions may transition respectively from the first region to the fourth region via the second and/or third regions on the respective first and second sides.

In another embodiment, the headgear comprises three pathways and wherein a first pathway extends in a fourth region, the second pathway extends in the first and second regions on a first side and the third pathway extends in the first and second regions on a second side.

The second pathway may extend below an ear on the first side and the third pathway may extend below an ear on the second side.

The second and third pathways may intersect. The intersection may be at a rear of the headgear.

The second pathway may extend from the second region on the first side to the third region on the second side and the third pathway may extend from the second region on the second side to the third region on the first side.

Tension lines disposed in the second and third pathways may be anchored to the positioning web at or adjacent to the location where the second and third pathways terminate.

The second and third pathways may terminate at a location adjacent to the fifth region on the second and first sides respectively. For instance, the second and third pathways may terminate above the ears on the second and first sides respectively.

The first pathway may extend in the fifth region.

The first pathway may extend rearwardly in the fourth region along the first side, along the fifth region from the first side to the second side and forwardly along the fourth region on the second side.

In this embodiment and in other embodiments, ends of the first and second pathway portions may be located in or adjacent to a respiratory interface region.

In embodiments including a pathway or pathway portion with an end at or adjacent to the respiratory interface region, the headgear may include a tension line-length controller in the interface region and a tension line disposed within the pathway or the first or second pathway portions may be linked to the controller to control the deployed length of tension line.

In embodiments of the headgear that include a fifth region and a pathway or pathway portion that commences or ends in the fifth region, the headgear may include a tension line-length controller in the fifth region and a tension line disposed within the pathway or first or second pathway portions may be linked to the controller to control the deployed length of tension line.

The respiratory interface may be a mask that is adapted to seal with the face of the user to communicate respiratory gas to the nose and/or mouth of the user.

The respiratory interface may be a mask that is adapted to seal with the nose and/or mouth of the user.

The respiratory interface may be an unsealed interface. That is, it does not form a seal with the user. For example, the unsealed interface may be cannula that are inserted into the nares of the user.

It is anticipated that the headgear described above works with the following interfaces to deliver an effective treatment: unsealed cannula, pillow masks, nasal masks, full-face masks, total face masks, and oral masks.

In the preceding passages and in the following description and claims, the terms “pathway” and “pathway portions” are references to any path forming formations for receiving a tension line. The term includes a continuous formation and includes discontinuous formations. Examples of discontinuous formations are a series of spaced apart locating members which collectively define a path for a tension line. Examples of locating members include bands, gates, hooks, loops, tubes, tunnels or guides.

The term “intersect”, and its grammatical variants, as used throughout the preceding passages and in the following description and claims in reference to the pathway or pathway portions is taken to mean that portions of the pathway or pathway portions merge, cross-over or overlap where the spacing between the pathway or pathway portions is zero. For example, in some embodiments, the spacing between pathway or pathway portions may be zero where the pathway or pathway portions occupy a common volume at an intersection. Alternatively, the pathway or pathway portions may remain separate where the spacing is zero due to the pathways crossing-over each other, i.e. the pathway or pathway portions are stacked on top of the other. In other embodiments, the pathway or pathway portions may be regarded as intersecting where the two tension-line portions occupy a single pathway or pathway portion. The two portions of the tension line passing through the single pathway or pathway portion may be parallel or may cross-over.

For convenience, the ‘controller’ is used interchangeably with the term ‘tension-line length controller’ throughout this specification.

### BRIEF DESCRIPTION OF THE DRAWINGS

Notwithstanding any other forms which may fall within the scope of the apparatus and method as set forth in the Summary, specific embodiments will now be described, by way of example only, with reference to the accompanying drawings in which:

FIG. 1 is an oblique view of user's head with a schematic outlined of an embodiment of headgear in an operative configuration (solid lines) in which the headgear is contracted to fit to the head of a user and in a donning and/or doffing configuration (dashed lines) in which the headgear is expanded for donning and doffing of the headgear.

FIGS. 2 and 3 are oblique front and rear views respectively of the headgear in FIG. 1 with an example of a pathway for a tension line.

FIG. 4 is a line-chart showing displacement of the positioning web and the tension line for each phase of donning, treatment (“pressure on”) and doffing.

FIG. 5 is a schematic force experienced by the user vs displacement graph showing force contributions of the tension line and the positioning web for donning, treatment and doffing without user adjustment of the tension line.

FIG. 6 is a schematic force experienced by the user vs displacement graph showing different force contributions of the tension line and the positioning web for the small, medium and large percentile head sizes.

FIG. 7 is a force experienced by the user vs accumulative change of displacement graph for each phase of donning, treatment and doffing.

FIG. 8 is a line-chart showing displacement of the positioning web and the tension line for each phase of donning, treatment (“pressure on”) and doffing with user adjustment of the tension line before activation of a flow generator.

FIG. 9 is a schematic force experienced by the user vs displacement graph showing force contributions of the tension line and the positioning web for donning, treatment and doffing with user adjustment of the tension line before activation of a flow generator.

FIG. 10 is a line-chart showing displacement of the positioning web and the tension line for each phase of donning, treatment (“pressure on”) and doffing with user adjustment of the tension line after activation of a flow generator.

FIG. 11 is a schematic force experienced by the user vs displacement graph showing force contributions of the tension line and the positioning web for donning, treatment and doffing with user adjustment of the tension line after activation of a flow generator.

FIG. 12 is a schematic view showing the effect of changing the length of the tension line in the pathway shown in FIG. 1.

FIGS. 13A, 13B and 13C are schematic front side and top views of a mask and tension line of the headgear according to the embodiment shown in FIG. 1.

FIG. 14 is a schematic view of a pathway about a patient's head according to the embodiment of headgear shown in FIGS. 2 and 3.

FIG. 15A is a rear oblique view of headgear that incorporates the pathway according to the embodiment shown in FIG. 14.

FIG. 15B is a cross-sectional view of the positioning web along the line II-II in FIG. 15A

FIGS. 16A and 16C are oblique views of a tension-line length controller which incorporates a tension line-length controller disposed on the front of the headgear in unlocked and locked positions, respectively, and FIGS. 16B and 16D are cross-sectional views of the combined controller in FIGS. 16A and 16C, respectively, without a coil spring.

FIG. 17 is a schematic view of a pathway about a patient's head according to another embodiment of headgear.

FIG. 18 is a schematic view of pathways path about a patient's head according to another embodiment of headgear.

FIG. 19 is a schematic view of pathways about a patient's head according to a further embodiment of headgear.

FIG. 20 is a schematic view of pathways about a patient's head according to another embodiment of headgear.

FIG. 21 is a schematic view of pathways about a patient's head according to another embodiment of headgear.

FIG. 22 is a schematic view of pathways about a patient's head according to another embodiment of headgear.

FIG. 23 is a schematic view of a pathway about a patient's head according to another embodiment of headgear.

FIG. 24 is a schematic view of a pathway about a patient's head according to another embodiment of headgear.

FIG. 25 is a schematic view of a pathway about a patient's head according to another embodiment of headgear.

FIG. 26 is a front oblique view of headgear that incorporates the pathway according to the embodiment shown in FIG. 25.

FIG. 27 is a schematic view of a pathway about a patient's head according to a variation of the headgear embodiment shown in FIGS. 25 and 26.

FIG. 28 is a schematic view of a pathway about a patient's head according to another embodiment of headgear.

FIG. 29 is a schematic view of pathways about a patient's head according to another embodiment of headgear.

FIG. 30 is a schematic view of pathways about a patient's head according to another embodiment of headgear.

FIG. 31 is a schematic view of pathways about a patient's head according to another embodiment of headgear.

FIG. 32 is a schematic view of pathways about a patient's head according to another embodiment of headgear.

FIG. 33 is a schematic view of pathways about a patient's head according to another embodiment of headgear.

FIG. 34 is a schematic view of pathways about a patient's head according to another embodiment of headgear.

FIG. 35 is a schematic view of pathways about a patient's head according to another embodiment of headgear.

FIG. 36 is a schematic view of pathways about a patient's head according to another embodiment of headgear.

FIG. 37 is a schematic view of pathways about a patient's head according to another embodiment of headgear.

### DETAILED DESCRIPTION

Embodiments will now be described in the following text which includes reference numerals that correspond to features illustrated in the accompanying Figures. To maintain clarity of the Figures, however, all reference numerals are not included in each Figure.

The descriptions of various embodiments of headgear which follow include reference to pathway locations in the context of the headgear being fitted to a user's head. That is, some description refers to pathways tracing, crossing or extending above, around or over various parts of a user's head. However, it is important to understand that the pathway is part of the headgear and the pathway retains the same path through the headgear regardless of whether the headgear is fitted to a user or not. The descriptions of the pathway relative to features of the head are provided to facilitate an understanding of the various pathway configurations and how one or more tension lines disposed within the different pathways carry tension and distribute tension through the positioning web (i.e. provide an understanding of the force experienced by a user). The description of the various embodiments is not intended to restrict the scope of the embodiments or the scope of the claims to headgear only when it is fitted to the user's head. The claims and the embodiments include within their scope headgear that is not fitted to the user because, as mentioned above, the pathway remains the same in the headgear whether it is fitted to the user or not.

The embodiments described below are described in the context of a sub-nasal full face mask and, therefore, in the context of a positioning web that is shaped to co-operate with a sub-nasal full face mask. It will be appreciated, however, that the headgear may incorporate alternative interfaces and the positioning web may have an alternative shape which fits with the given alternative interface to provide the same effect, including non-sealing interfaces such as non-sealing cannula for high flow therapy. That is, the pathway may be the same or similar despite the interface having a different form and fit to the user's face. The terms “mask”, “interface” and “respiratory interface” as used throughout the description and claims are not limited to sub-nasal masks and instead extends to other interfaces which form a generally gas-tight seal for delivering pressure therapy or other respiratory therapies via the mouth, the nares or both.

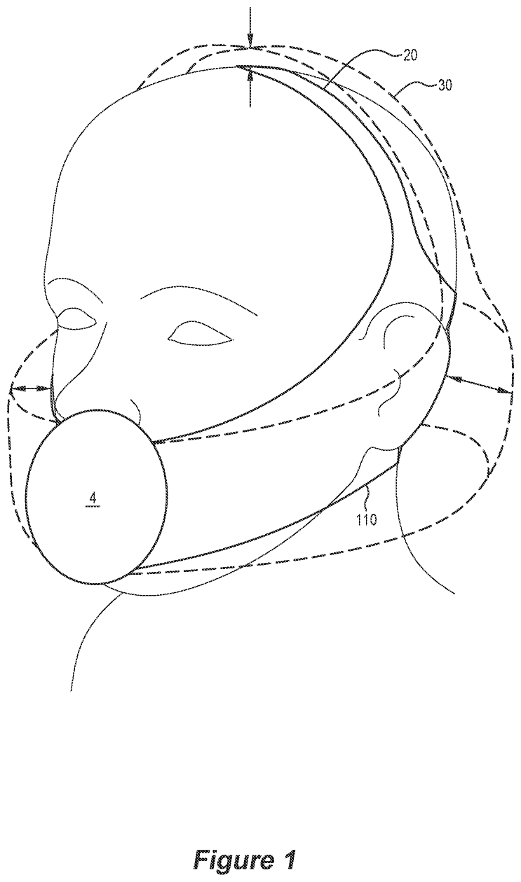

An example of headgear 110 is shown in FIGS. 1 to 3 as comprising a positioning web 112 with a pathway 114 for a tension line 150, a respiratory interface 4. The headgear 110 further comprises a tension-line length controller 140 which controls the length of tension line 150 that is deployed in the pathway 114.

### Positioning Web

The positioning web 112 is expandable and contractible. More specifically, the positioning web 112 is expandable to adopt an expanded configuration 30 (denoted by the dashed lines in FIG. 1). In the expanded configuration 30 the positioning web 112 is dimensioned to fit over the user's head so the positioning web 112 can be donned or doffed. The positioning web 112 is contractible from the expanded to an operative configuration 20 (denoted by solid lines in FIG. 1) where the positioning web 112 is fitted to the head of a user.

In the illustrated embodiment, the positioning web 112 is formed from an elastic material. As explained in more detail below, the elasticity of the positioning web 112 is relatively low so that the positioning web 112, in the operative configuration 20, is comfortably and snugly fitted to the user's head.

While the embodiment shown in FIG. 1 involves the positioning web being formed of an elastic material, it is also to be appreciated that the positioning web may not be elastic but may be mechanically expandable and contractible. The mechanical expansion and contraction may be in the length direction, may be in the height direction or may be in both the length direction and the height direction. With a mechanically expandable and contractible positioning web, the positioning web may be formed of elastic material or substantially inelastic material or may be formed of material with anisotropic elasticity. For example, when the positioning web is formed of relatively inelastic material, the positioning web may have telescopically extending portions or portions on rails that slide over each other to expand and contract the positioning web.

In another example, the mechanical expandability may comprise two or more overlapping sections of positioning web which are translatable relative to each other to permit expansion and contraction of the positioning web. The overlapping sections may be separated by folds in the positioning web. In an example of a positioning web with anisotropic elasticity, the positioning web may be elastic in the height direction and may be relatively inelastic, but mechanically expandable and contractible, in the length direction. In another variation of the positioning web with anisotropic elasticity, the positioning web may be elastic in the length direction and may be relatively inelastic, but mechanically expandable and contractible in the height direction. Alternatively, the position web may comprise relatively inelastic portions and mechanically expandable and contractible portions. For example, the one or more pathways or pathway portions may be relatively inelastic and may be connected to spaced apart panels which are mechanically expandable and contractible.

The operative configuration 20 has sufficient tension throughout the positioning web 112 to hold a respiratory interface 4 in contact with a user's face when coupled to a respirator device, but insufficient tension to counteract a blow-off force or a tube pull force. The tension level is much lower than a force required to counteract a blow-off force in order to make the positioning web comfortable to wear. During use, the positioning web 112 remains in the operative configuration 20 and, therefore, continues to impart roughly the same tension contribution to holding the headgear 110 in position. That is, the positioning web 112 continues to impart the same low level of pressure to the user's head during use so the user's comfort levels attributable to the positioning web 112 remain roughly the same during use. The tension in the positioning web 112, on account of the tension lines 150 counteracting variable blow-off and tube-pull forces, may vary during use as it acts to retain the tension line 150 in position about the user's head.

The elasticity of the headgear may be anisotropic. This may take the form of one or more regions of the positioning web 112 having an elasticity that is different to the elasticity of other regions. In particular, the elasticity of the positioning web may be different in the height-direction of the positioning web compared to the elasticity in the length-direction of the positioning web.

In some embodiments, the elasticity of the positioning web may be greater in the length-direction than the elasticity in the height-direction. Furthermore, the elasticity in the height-direction may be selected to resist buckling and folding in the height-direction. The combination of lower elasticity in the height-direction and higher elasticity in the length-direction enables the headgear to be readily expandable for donning and doffing and, at the same time, to resist folding and buckling in the height direction.

The different elasticities in the length-direction and the height-direction may be provided by differences in construction between the regions, differences in materials in the regions or a combination of both.

In addition to enabling the headgear 110 to be donned and doffed readily, the elasticity of the positioning web 112 contributes to maintaining the position or spacing of the pathway 114 in the height direction, and therefore the position or spacing of the tension line 150, in the operative configuration. There is a tendency for the tension line 150 to be pulled from one location to another location owing to the curvature or contours of the head. For example, a single tension line 150 fixed to an interface 4 extending about the user's head and passing over the crown of the head will tend to slide forwardly over the user's head when tension is applied. The tendency increases with increasing tension. Given the relatively high tension in the tension line 150 when the headgear is in use, the positioning web 112 serves to retain the tension line 150 in the required position because the tension vector is directed into the positioning web 112 where it is counteracted by the elasticity of the positioning web 112 and by an opposing tension vector associated with the tension line 150 in another region of the positioning web 112. To be more specific, the tension vector directed into the positioning web 112 from the tension line 150 in a first portion of the pathway 114 is counteracted to an extent by an opposing tension vector which is directed into the positioning web 112 from the tension line 150 in a second portion of the pathway spaced apart across the height dimension of the positioning web 112 from the first portion of the pathway 114. This means that the positioning web 112 acts to stabilise the positioning or spacing of the tension line 150 about the user's head at least partly because the counteracting effect of forces transferred into the positioning web 112 by the spaced tension lines 150. For example, if one part of the tension line 150 is located above a nominal equator line about the user's head (denoting the greatest circumference of the head) and another part of the tension line 150 is located below the equator line, the tension in the upper part will impart a force that pulls the positioning web 112 toward the top of the head while the part of the tension line 150 below the equator will impart a force that pulls the positioning web toward the user's neck. The two opposing forces act against each other and contribute to retaining the tension line 150 in position during treatment.

For example, in the example shown in FIGS. 1 to 3, the tension line resides in a pathway 114 that runs along the upper and lower rims 132 and 134 of the positioning web 112. The tension line in the upper rim 134 will pull the positioning web upwardly owing to the contours of the head at the location of the upper rim 134. However, this pull is counteracted by a downward pull caused by the tension line in the lower rim 132 owing to the contours of the lower jaw and the inward tapering toward the neck from the base of the skull. The dynamics of the forces imparted to the positioning web 112 by the tension lines 150 will vary depending on the location of the pathway 114 about the user's head. That is, the contours of the user's head over which the pathway 114 passes will affect the extent to which tension is transferred from the tension line 150 into the positioning web 112.

An additional effect of transferring tension from the tension line 150 into the positioning web 112 is that the pressure applied to the user's head is less concentrated along the line of the pathway 114. Reducing the pressure concentration along the pathway 114 will make the headgear 110 more comfortable during treatment and therefore users are more likely to persist with the treatment.

### Pathway

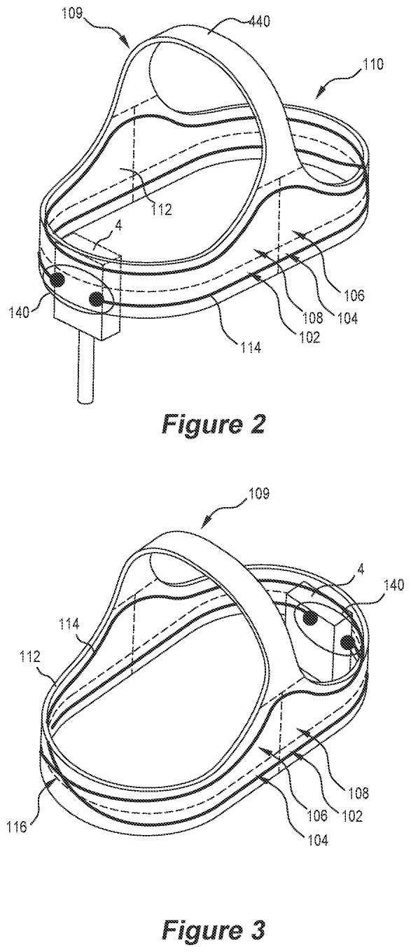

For the purpose of describing the pathway 114, the headgear 110 is notionally divided into 5 regions as shown in FIGS. 2 and 3. A first region 102 is at a front lower part of the positioning web, a second region 104 is at a rear lower part, the third region 106 is at a rear upper part above the second region 104 and the fourth region 108 is at a front upper part above the first region 102. The headgear 110 further includes a fifth region 109 in the form of a strap 440, that passes over the top of the user's head when the headgear is fitted.

While the fifth region 109 is shown as part of the positioning web 112 in FIGS. 1 to 3, the fifth region 109 may be formed as a separate component which is attachable in other examples. Either way, it should be appreciated that the fifth region 109 is an optional part of the positioning web 112.

The pathway 114 (FIGS. 2 and 3) extends at least part way along the positioning web 112 and is adapted to receive the tension line 150. The example shown in FIGS. 2 and 3 includes a single pathway 114 in the form of a sleeve. The pathway 114 extends, from a location at or adjacent the respiratory interface 4, along the left side of the headgear 110 in first and second regions 102, 104 and then extends along the right side in third and fourth regions 106, 108, across the respiratory interface and along the left side in the fourth and third regions 108, 106 and then along the right side in the second and first regions 104, 102 before ending at or adjacent to the respiratory interface 4.

The single pathway 114 makes two loops about the positioning web 112 and in doing so the spacing of the pathway 114 varies in the height-direction along the positioning web 112.

### Tension Line

A tension line 150 is disposed within the pathway 114 and has ends outside the pathway 114 that are linked to the controller 140 (described in more detail below) which controls the length of tension line 150 that is deployed in the pathway 114. The tension line 150 is inelastic compared to the expandability and contractibility of the positioning web 112. Additionally, the tension line 150 is substantially inextensible compared to the positioning web 112 at the tensions associated with blow-off and tube-pull forces. In other words, the tension line 150 is substantially inelastic at operating tensions of the tension line 150. However, the one or more tension lines may be sufficiently elastic to stretch to account for small changes in tension at the operating tensions of the tension line. In such embodiments, the deployed length may not change when small changes in tension occur, but instead the tension line stretches (or retracts) in response to the change in tension. These small changes in tension might occur, for example, due to the user adjusting their head position during sleep.

The tension lines may have an elastic modulus that is at least 120% or 150% or 200% or 300% or 400% of the elastic modulus of the positioning web.

In terms of permissible strain during use, the tension lines may exhibit an elastic strain that is less than 5% or 4% or 3% or 2% or 1% at tensions associated with blow-off and tube-pull forces. The tension lines may exhibit the strain at tensions of up to 50N, preferably up to 40N, preferably up to 30N, more preferably up to 20N.

Preferably, the tension line 150 is inelastic for tensions of at least double the blow-off and tube-pull forces. For example, the tension line 150 is substantially inelastic under a tensile force of up to 50N or up to 40N or up to 30N or up to 20N.

The tension lines 150 may have an elastic strain that varies along the length of the tension line 150 for a given tension applied to the tension line 150. The variation in the elastic strain may result from the tension line 150 comprising a plurality of sections and the elastic strain of at least one section differing from the elastic strain of another section for a given tension applied to the tension line 150. The elastic strain of the section or sections forward of the user's ears may be less than the elastic strain of the section or sections rearward of the user's ears.

Sections with a higher elastic strain than the elastic strain of other sections may exhibit elastic strain greater than 5% while the tension line 150 exhibits an overall elastic strain that is less than 5%. The overall elastic strain may be less than 5% at tensions associated with blow-off and tube-pull forces.

The differing elastic strain in the tension line 150 may be due to the tension lines 150 having an elastic modulus that varies along the length of tension line 150. In one form, the tension line 150 may comprise a plurality of sections and the elastic modulus of at least one section differs from the elastic modulus of another section. Furthermore, the elastic modulus in the section or sections forward of the user's ears may be greater than the elastic modulus of the section or sections rearward of the user's ears.

Sections with a lower elastic modulus than the elastic modulus of other sections may exhibit elastic strain greater than 5% while the tension line 150 exhibits an overall elastic strain that is less than 5%. The overall elastic strain may be less than 5% at tensions associated with blow-off and tube-pull forces.

The variation in elastic strain or elastic modulus may be due to the tension line 150 having a composition that varies along the length of the tension line 150. The one or more tension lines 150 may have a composition forward of the user's ears that has a higher elastic modulus than an elastic modulus of a composition of the tension line 150 rearward of the user's ears.

Alternatively, the variation in elastic strain or elastic modulus may result from the tension line 150 having a cross-sectional area that differs along the length of the tension line 150. This may involve at least one section of the tension line 150 having a cross-sectional area that differs from the cross-sectional area of other section. Alternatively, the tension lines 150 may comprise an assembly of different materials. In this alternative, the sections of the tension line 150 may comprise different materials.

The tension line 150 may take the form of a woven or non-woven filament, line, thread, ribbon or tape. In one embodiment, the tension line is a small gauge fishing line that is rated to handle tension forces that are at least double the anticipated blow-off force. However, the term “tension line” should not be interpreted as being limited to those structural forms.

### Tension Line Intersection

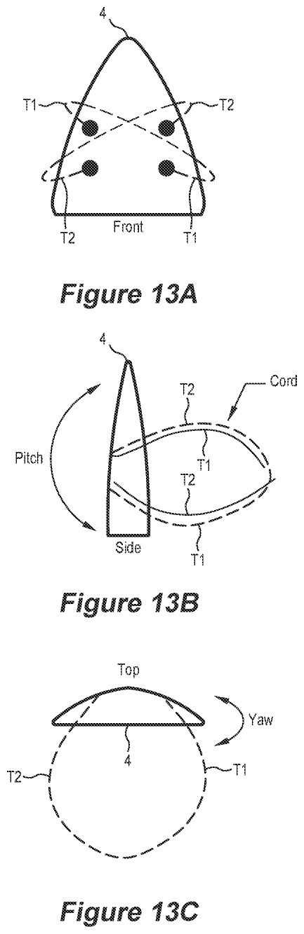

At the rear of the positioning web, first and second portions of the pathway intersect so the spacing at this point is zero. This means that tube pull or a user pushing the side of their face into a pillow re-distributes the tension in the tension line so that the interface 4 is retained on the user's face in a working position, i.e. the chances of excessive leakage or blow-off are reduced. Specifically, when the interface 4 is pulled to one side, the tension in the tension line 150 increases on the opposite side to resist the force. That tension is transferred to the same side as the pulling force so the tension remains in the tension line 150 on that side, rather than the tension reducing to a point where the seal on that side of the interface 4 is compromised. This effect is created by linking the tension line (T1 and T2 in FIGS. 13A to 13C, for example) from one side of the interface 4 to the other side via the rear of the user's head. The effect also works in the vertical direction on account of crossing the tension line 150 over itself at the intersection 116. In FIGS. 2 and 3, this means that tension in the tension line 150 running along the lower left side of the headgear 110 is transferred to the tension line running along the upper right side of headgear on account of the pathway 114 transitioning from below the left ear to above the right ear in passing about the rear of the head. In FIG. 13A, this is shown as T1 connecting to the interface 4 at the lower left side of the mask and the upper right side. The same applies to tension line running along the lower rights side and the upper left side of the headgear 110 in FIGS. 2 and 3 and is shown in FIG. 13A as T2 connecting to the mask at the upper left side and the lower right side.

The overall effect of the tension line cross-over is that angular rotation (i.e. pitch and yaw as shown in FIGS. 13B and 13C) of the interface 4 is controlled. This makes the headgear 110 more dynamically responsive to tube pull and interference from pillows when changing sleeping positions.

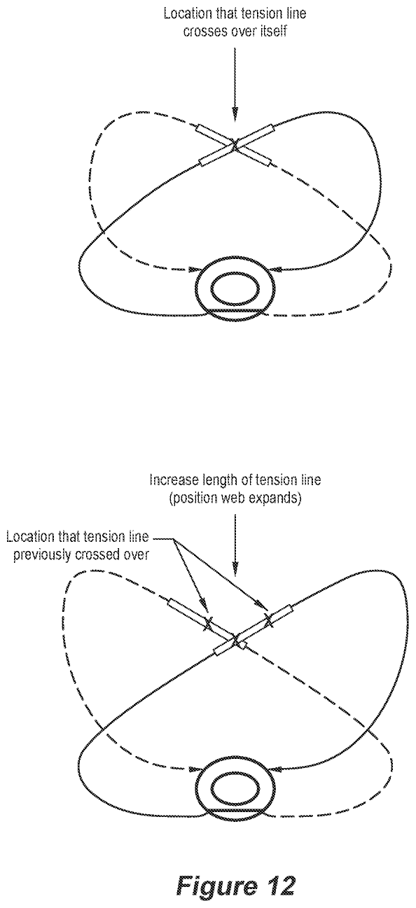

Another effect of the tension line 150 crossing over is that the position of the cross-over along the tension line changes when the length of the tension line in the pathway 114 is adjusted by the controller 140 or when the controller 140 is unlocked and the positioning web 112 is expanded and contracted during donning and doffing. Using the images in FIG. 12 as an example, the tension line crosses over itself at an initial cross-over point (marked as an “X” at the end of the dashed arrow in the upper image). Increasing the length of the tension line in the pathway 114, such as when the controller 140 is unlocked and the positioning web 112 is expanded for donning and doffing, tension line 150 is transferred from the controller 140 into the pathway 114 to increase the deployed length of tension line 150 in the pathway 114. While the location of the cross-over point relative to the user's head remains the same, the location of the cross-over along the tension line 150 changes. As shown in the lower image in FIG. 12, dashed arrows point to the former location of the cross-over on the tension line 150. It follows that the position of the cross-over location varies when the length of the tension line 150 in the pathway 114 is adjusted.

### Tension-Line Length Controller

The controller 140 is operable to control the length of tension line deployed to the or each pathway 114. The controller 140 fixes the deployed length of tension line in the pathway 114, thereby preventing expansion of the positioning web 112. The controller 140, for example, can hold the positioning web 112 in the operative position when the headgear has been donned. It can also be operable to deploy the tension line 150 into the pathway 114 to permit expansion of the positioning web to the expanded configuration for donning and/or doffing. In some embodiments, the controller 140 may be operable to adjust the length of deployed tension line 150. Such adjustment may involve winding-in or paying-out the tension line.

The controller controls the length of tension line that is deployed in the pathway. This deployed length may limit the expansion of the positioning web, and subsequently minimise the risk of overloading the positioning web 112. Thus, the controller acts as a lock-out mechanism for the positioning web 112. Such a lockout mechanism would both maintain durability of the positioning web and control the lockout distance of the headgear assembly efficiently.

The controller 140 may take any suitable form for controlling the length of the tension line 150 that is deployed into the pathway 114. Some examples of suitable controllers 140 are disclosed in PCT publication WO2014/025267 in the name of Fisher & Paykel Healthcare Limited. The disclosures in that PCT publication are incorporated herein by this reference.

### Operation of the Headgear

The respiratory interface 4 is associated with a front part of the positioning web such that when the positioning web is fitted, the respiratory interface 4 is positioned to interact with the mouth and/or nares of a user. The respiratory interface 4 needs to be held against the face to create and maintain a substantially air-tight seal. In many treatments, however, the gas pressure of the gas supplied to the respiratory interface 4 increases and decreases through the respiratory cycle, meaning that the headgear 110 needs to counteract at least a variable blow-off force to maintain the seal through the respiration cycle. Balancing this force (and any tube-pull force) is the primary function of the headgear 110 and a balanced fit is achieved when the counteraction forces in the headgear 110 substantially match the blow-off and tube-pull forces.

An elastic positioning web 112 allows for a quick and easy fit to hold the respiratory interface 4 on the user's face when the respiratory interface 4 is not in use because the elasticity of the positioning web 112 does not provide the minimum force required to counteract the blow-off force. However, contraction of the positioning web 112 when the headgear 110 is fitted reduces the length of the pathway and therefore the length of the tension line in the pathway decreases. Fixing the deployed length with the lock at this point means that the tension line bears zero or close to zero tension and, therefore, the force experienced by the user is the elastic contraction force of the positioning web and a negligible contribution from the tension line. In other words, the resting on head (non-use) state of the headgear 110 does not apply a force that is equivalent to the force required to counteract the minimum blow-off force in and, therefore, the headgear 110 is not subject to over tightening and excessive forces, which can occur with manually adjusted and elasticated headgear.

An inelastic positioning web 112, however, doesn't impart any force on the user outside of the treatment phase on account of the mechanical expandability and contractibility. In the absence of an elastic force contributed by the positioning web 112 to hold the headgear 110 in position, the deployed length of tension line 150 is reduced until the positioning web 112 is snuggly fitted. The positioning web 112 adopts the contoured shape of the head so that the headgear is held in position by the tension line 150 which prevents expansion of the position web to a size that it can be freely removed from the user's head.

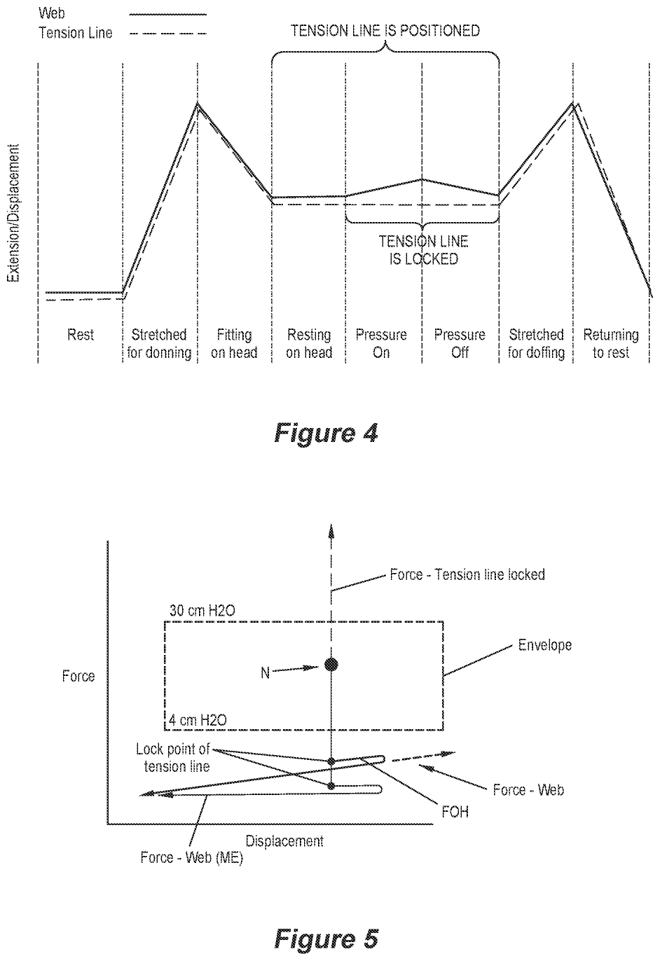

The forces imparted on a user during donning, doffing and in-use are outlined in more detail in FIGS. 4, 5 and 7.

FIG. 4 shows the displacement of the positioning web 112 and the tension line 150 through the process of donning the headgear 110, fitting it, using the headgear 110 for a treatment and then doffing the headgear 110. The displacement of the positioning web is shown in two forms, namely “Web” to denote an elastic positioning web 112 and “ME” to denote a mechanically expandable and contractible positioning web 112)

Prior to donning, the headgear 110 is in a relaxed or “rest” state whereby the positioning web 112 is fully contracted, or at least partially contracted compared to the expanded state, given that there is no force applied to the positioning web 112 causing it to expand. This is shown as the “Rest” region in FIG. 4 and is denoted by zero displacement. The donning process is initiated by expanding the positioning web 112 to fit the user's head. This is denoted in FIG. 4 as the “Stretched for donning” phase and it involves, if the controller 140 is locked, unlocking the controller 140 so that the tension line 150 can transition freely into the pathway 114, and then expanding the positioning web 112 so that its length is greater than the circumference of the user's head in order to fit the positioning web 112 over the user's head. Expanding the positioning web 112 causes the length of the pathway 114 to increase, thereby drawing more tension line 150 into the pathway 114 which is why the tension line 150 is also subject to a positive displacement in this stage. During this phase, the positioning web is in an expanded configuration. In the absence of any resistance to displacement of the tension line 150 (the tension line 150 may contribute some force but it is typically so small that it is negligible), the user will generally experience force only from the elasticity of the positioning web 112 where the positioning web 112 is elastic. That resistance can be roughly equated to an ideal spring, so the resistance (or pressure force experienced) is a function of the displacement, i.e. F=kx. This is shown as region 1 in FIG. 7 where the resultant force experience by the user is denoted as “F” and the x-axis represents cumulative displacement (i.e. expansion and contraction are both treated as positive displacement and are aggregated along the x-axis). Where the positioning web is mechanically expandable and contractible, the user will generally experience no force, which is represented by the Force Web-ME graph in FIG. 5 which shows that the force does not increase during expansion of the positioning web 112 from the “Rest” state to the expanded configuration.

Once the positioning web 112 is expanded to the expanded configuration for fitting, the headgear 110 is then placed over the user's head and is allowed to contract to a length which matches the circumference of the user's head. This is represented in FIG. 4 as the reduction in displacement associated with the step of “Fitting on head”. At this point, the positioning web 112 is in its operative configuration. The reduction in displacement during contraction is less than the expansion during stretching because the resting length of the positioning web 112 is shorter that the circumference of the user's head. The result is that, with the elastic positioning web 112 expanded less, it will impart a smaller tension force on the user's head than the tension in the positioning web 112 in the expanded configuration and this is represented in FIG. 7 by a reduction in experienced force in region 2. This force, as described previously will be enough to hold the interface 4 on the user's head, but is not enough to counteract even the minimum blow-off force. The contraction of the positioning web 112 results in shortening of the pathway 114 and a corresponding reduction in displacement of the tension line 150, meaning that the length of tension line in the pathway 114 reduces.

By way of contrast, contracting an inelastic positioning web 112 to fit the user's head involves reducing the deployed length of tension line 150 to cause mechanical contraction. Again there is generally no force experienced by the user during the contraction because, up until the positioning web 112 is fitted, the positioning web 112 is greater in the length direction the circumference of the user's head.

Once fitted, the headgear 110 can rest on the head with the interface 4 held in position while there is no pressurised respiratory gas supplied to the interface 4. Pressurised respiratory gas may be provided by any suitable source, but for the purposes of describing the headgear 110 here, reference will be made to a flow generator as the source of respiratory gas. It will be appreciated, however, that the headgear 110 is not limited to use with a flow generator and may be used with other sources of pressurised respiratory gas. This is represented in FIG. 4 as the “Resting on head” phase where the displacement does not change. This means that the forced experienced by the user remains constant because only the positioning web 112 is imparting any force on the user. This phase is represented by region 3 in the force curve shown in FIG. 7. During each of these phases (i.e. phases 1, 2 and 3 in FIG. 7), the controller 140 is unlocked so the tension line does not carry any tension. The only force experienced by the user is the spring force (F=kx) attributable to the positioning web 112.

In preparation for treatment, the controller 140 is locked to fix the deployed length of the tension line 150 (i.e. fix the displacement) in the pathway 114. Fixing the displacement of the tension line 150 means that expansion of the positioning web 112 is restricted because the blow-off force and the tube pull force is counteracted by the tension line 150 running through the pathway 114. The tendency for the blow-off force to push the interface 4 off the user's face is resisted by the fixed length of tension line 150 which passes about the user's head.

When the flow generator is activated, the gas pressure supplied to the interface 4 creates a blow-off force and, when the flow generator is deactivated, the blow-off force is removed. These actions are denoted in FIG. 4 by the “Pressure on” and “Pressure off” phases and in FIG. 7 by phases 4 and 6 respectively. During these phases, the displacement in the tension line 150 remains the same until the controller 140 is next unlocked. However, the tensile forces are distributed through the positioning web 112 in an effect called “force vectoring”, when the gas pressure increases to the treatment pressure (this is also shown in the corresponding phases in FIG. 4). The displacement of the positioning web 112 under force vectoring is very small to negligible owing to the tension line 150 largely isolating the positioning web 112 from extension during operation of the flow generator. This means that the contribution by the positioning web 112 to the force experienced by the user is small during use of the flow generator.

The force vectoring, as described above, results from a transfer of tension in the tension line 150 to tension across the height dimension of the positioning web 112 in the spacing between the between two portions of a pathway 114 or between pathways 114. This is due to the tension line 150 pulling over a curved surface (i.e. the patient's head) because, without the positioning web 112, the tension line can only hold tension along the line of the tension line 150. For variable pressure treatments (i.e. when the inspiratory and expiratory pressures provided by the flow generator are different from each other and/or change over the treatment period), the transfer of tension into the positioning web 112 will increase and decrease with the increase and decrease in counteracting force in the tension lines 150 and will result in very slight expansion and contraction of the positioning web 112 in the height dimension. Although this displacement is typically so small that it makes a negligible contribution to the force experienced by a user.

With the controller 140 locked, the tension line 150 provides a reactive tension force which counteracts the blow off-force, any tube pull force and, to a small extent, tension in the positioning web 112 as described below. The tension in the tension line 150 is, therefore, approximately equal to the blow-off force plus any tube pull force (i.e. Tension=PA+T_{PULL}). This tension force is represented as phase 5 in FIG. 7.

For treatments involving variable pressure supply, such as a high pressure supply of respiratory gas during inhalation and a low pressure supply during exhalation, the blow-off force will vary depending on the gas pressure in the interface 4. As mentioned above, this variation in the blow-off force is counteracted by locking the controller 140 so that the tension line 150 remains at a fixed length during the treatment.

After treatment, the flow generator is deactivated and the force experienced by the user drops back to the force which the positioning web 112 applies for its given displacement because the tension associated with the blow-off force is removed when the flow generator is deactivated. As mentioned previously, this force attributable to the positioning web 112 is a relatively low force as indicated by phase 7 in FIG. 7. The displacement after the flow generator is de-activated is the same as the displacement when the headgear is fitted, as represented in the “Resting on head” phase in FIG. 4. In this circumstance, the tension line 150 makes no contribution, or a negligible contribution if any at all, to the force experienced by the user.

To doff the headgear 110, the controller 140 is unlocked, so that any remaining tension in the tension line 150 is released and so that the positioning web 112 can be expanded. The user then expands the positioning web 112 to a displacement which enables removal of the headgear 110 from their head (i.e. to the expanded configuration). This is typically done by sliding the headgear off over their head. This expansion is represented in FIG. 4 as the “Stretched for doffing” phase and the corresponding force (which is the spring force associated with expanding the positioning web 112) is represented in FIG. 7 as phase 8. As the controller 140 is unlocked, stretching of the positioning web 112 will draw the tension line 150 into the pathway 114. However, the force applied by the headgear 110 is mostly attributable to the positioning web 112. If any force is attributable to the tension line 150 at all, it is small or negligible.

As the headgear 110 is removed, the displacement of the positioning web 112 will decrease. For an elastic positioning web 112, the decrease in displacement is a contraction of the positioning web 112 due to its elasticity. The displacement decreases until the positioning web 112 reaches its “Rest” state (i.e. zero displacement). This is represented in FIG. 4 as the “Returning to rest” phase. As the displacement returns to zero, the force attributable to the positioning web 112 also decreases to zero as shown in phase 9 in FIG. 7. It will be appreciated that FIG. 7 is an idealised representation of the force and displacement and in reality, if the headgear 110 is worn for long periods of the time, the positioning web 112 will largely return to the “rest” state when it is removed. Accordingly, the force, as represented in FIG. 7, will return to zero.

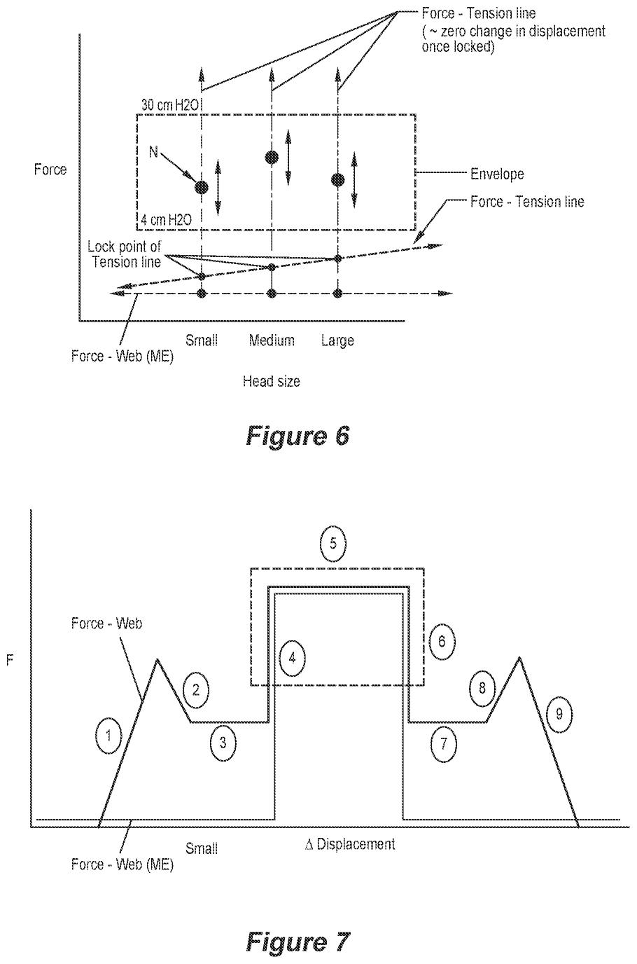

Another way of appreciating the force experienced by a user during donning, doffing and during treatment is to consider the respective contributions of the positioning web 112 and the tension line 150. FIG. 6 shows a graph of displacement versus force where the force contributed by the web is denoted by the dashed line “Force-Web” and the force attributable to the tension line is denoted by three separate dashed lines “Force-Tension line”.

During donning and doffing of a positioning web 112 that is elastic in the length direction, the positioning web 112 makes the only contribution to the force experienced by the user and that force is the spring force (i.e. F=kx). The slope of the “Force-Web” line shows that the force increases and decreases proportionally with displacement. Accordingly, the force increases from the “Rest” state (FIG. 4) as the positioning web 112 is expanded and then contracts slightly once fitted. The tension line 150 is able to slide through the pathway 114 prior to locking, so the primary forces are from the positioning web 112, with only a small contribution, if any, from the tension line 150. The “fitted” point on the Force-Web line is shown as the intersection point between the “Force-Web” line and the vertical “Force-tension” line and will be at different displacements for different head sizes. As examples, fitting points for three different head sizes generally representing small, medium and large head sizes are shown in FIG. 6. As described above, once fitted, the tension line 150 is locked (denoted in FIG. 6 as “Lock point of tension line”) so the displacement of the tension line 150 and the positioning web 112 remain the same as at the “fitted” point during the treatment. For simplicity, force variations associated with the tension transfer into the positioning web 112 are not shown in all figures.

After the tension line 150 is locked, the flow generator is activated and a blow-off force is created. The tension line 150 counteracts the blow-off force and any tube pull force but its displacement is fixed, so the force increases along the “Force-Tension line” into an operating envelope (denoted by the dashed rectangle). During the treatment, the tension line 150 carries most of the force and, with the tension line 150 fixed, variations in blow-off force or any tube pull forces are counteracted by tension in the tension line 150. This means that variations in the flow generator gas pressure, such as the flow generator supplying respiratory gas in line with the inhalation and exhalation cycle, are met with variations in tension in the tension line 150. The same applies to variations in the tube pull force, so the user only experiences a force that is equivalent to the instantaneous total of the blow-off force, any tube pull force and the force attributable to the positioning web 112. This variation is denoted in FIG. 6 by the double-ended arrows alongside the Force-Tension lines to show a force range which the user may experience during treatment. For convenience, this force range is also denoted in FIG. 6 as nominal force N. The same nominal force N is used (instead of double ended arrows) in FIGS. 5, 9 and 11 to indicate a force range which the user may experience during treatment.

The process described as above of donning and doffing the headgear 110 is shown in FIG. 5 in terms of force versus displacement. During donning, the positioning web 112 is expanded to the expanded configuration, i.e. to a size that fits over the user's head (which is shown as the initial solid portion of the Force-Web line). Once it is positioned over the head, it is allowed to contract until it fits snugly about the head—the operative configuration. This contraction is the same contraction that is represented in FIG. 4 as the “Fitting on head” phase. In FIG. 5, this contraction would return along the same initial solid portion of the Force-Web line so the same force applies for a given displacement, however, for clarity it is represented as a U-turn and solid line portion FOH which is adjacent and parallel to the initial portion. In this contracted state, the positioning web 112 is at the “Resting on head” phase in FIG. 4 which is represented in FIG. 5 as the “Lock point of Tension line”. The controller 140 can now be locked to prevent any change in the displacement of the tension line 150. Now when the flow generator is activated, the tension in the tension line 150 increases to counteract the blow-off force and which force increase is represented as a solid portion of the Force-Tension line to the nominal force N. The treatment continues with the force at or around nominal force N, but any change in the force is not accompanied by a change in displacement, as shown by the Force-Tension line.

At the end of the treatment, the flow generator is deactivated so there is no longer any force attributable to the tension line 150 and, therefore, only the positioning web 112 contributes to the force so the force drops back to Force-Web line. At this point the controller 140 is unlocked and the headgear 110 is removed by expanding it to the expanded configuration (i.e. increasing displacement along the portion FOH) and then, once removed, allowing it to contract back to its resting state (along the initial solid portion of the Force-Web line).

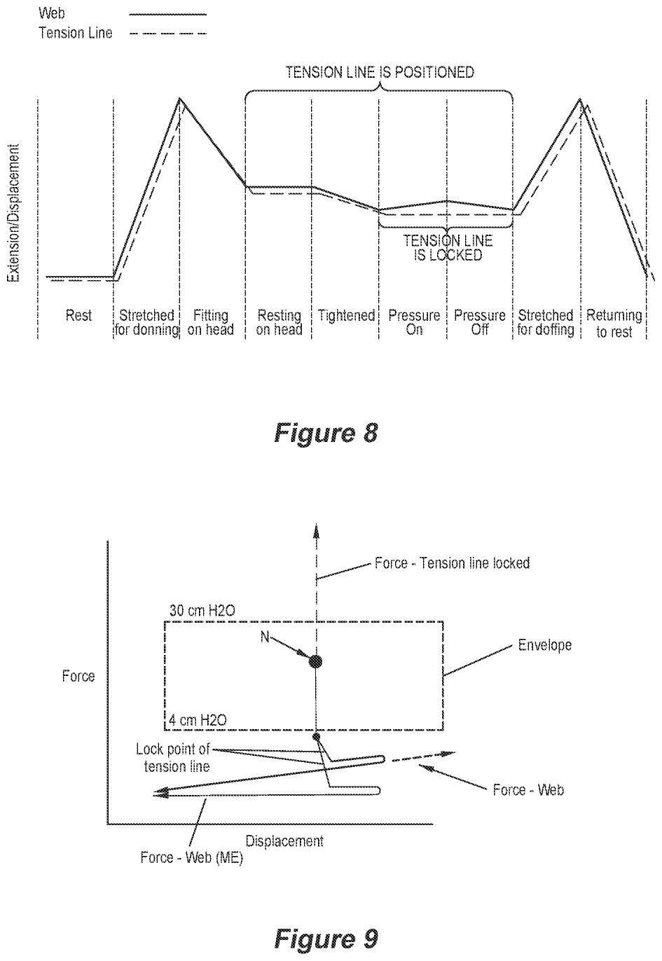

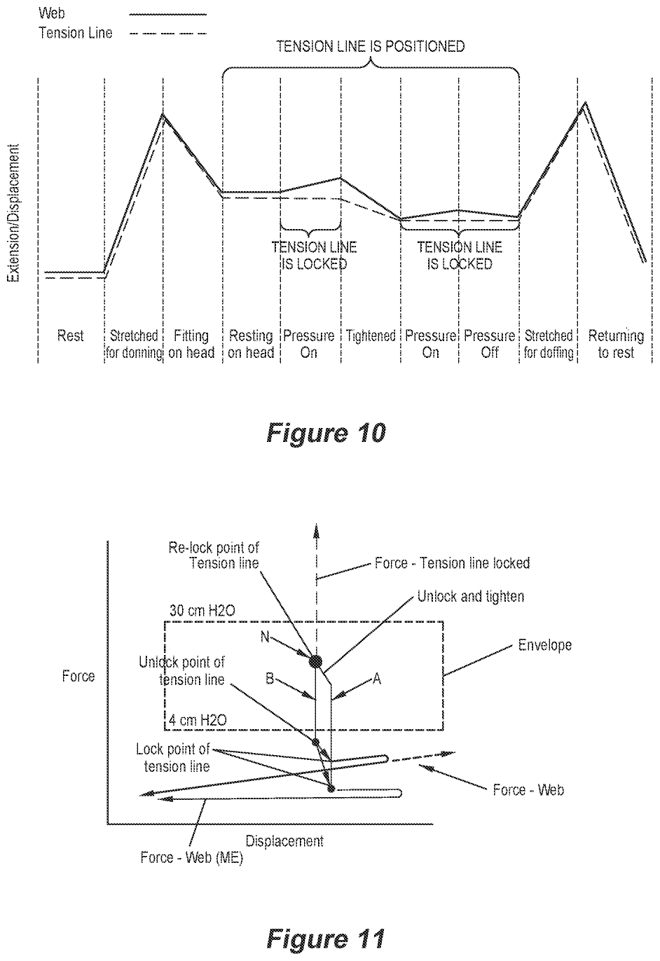

It is anticipated that some users will prefer to make the interface 4 feel more securely fitted to their face by reducing the deployed length of the tension line 150 to slightly tighten the headgear 110. This tightening is denoted by a reduction in the displacement of the tension line 150 in the “Tightened” phase of FIG. 8 and an increase in the force experienced by the user. This adjustment option is described in more detail below. It will be appreciated that tightening the tension line 150 will reduce the length of the positioning web 112 about the user's head, so there is a commensurate reduction in the displacement of the positioning web 112 and this is also represented in the “Tightened” phase in FIG. 8. While FIG. 8 shows that the headgear 110 may be “Tightened” before the flow generator is activated, another option involves tightening after the flow generator is activated. This option is shown in FIG. 10 and is described in more detail below.

Tightening is optional and is done based purely on user preference because a relatively soft pressure for holding the interface 4 onto the user's face prior to treatment commencing doesn't impact on the effectiveness of the headgear and the treatment once the controller 140 is locked and the flow generator is activated.

The initial stages of fitting so the positioning web 112 snugly rests on the user's head are the same with this option as described above. This option differs, however, in that before locking the controller 140, the user reduces the deployed length of tension line (i.e. decreases displacement in) a small amount (represented as the “Tightened” phase in FIG. 8) and, in doing so, increases a tension force in the tension line 150 from zero to an amount of force where the user feels that the headgear 110 is securely fitted. This increase in force is represented in FIG. 9 as the “User adjusted force”. For a mechanically expandable and contractible positioning web 112, the process is the same for donning and user adjustment, but the positioning web doesn't make a contribution to the force experienced by a user. This means that the user adjustment increases the force experienced by the user from roughly zero force to the amount of force where the user feels that the headgear 110 is securely fitted. The change in the “User adjusted force” for the mechanically expandable and contractible positioning web 112 is greater than the change in the “User adjusted force” for the elastic positioning web 112 (Force-Web) in FIG. 9. Along with the force increase, the displacement decreases from the point where the positioning web 112 rests on the user's head (i.e. the displacement is commensurate with the distance about the user's head in the length direction) to a point where the headgear feels securely fitted.

From the point that the user adjustment is completed, the controller 140 is locked and pressurised respiratory gas is supplied to the interface 4 (represented as “Pressure on” in FIG. 8 and as the force in the tension line rising to the nominal force N in FIG. 9. In other words, the treatment is applied in the same way as described above in respect of FIG. 6. When the treatment is ended (represented as the “Pressure off” phase in FIG. 8), the force decreases to the same level it was at before the flow generator was activated (represented as the “Lock point of Tension line” in FIG. 9), namely there is a small force contribution from tension in the tightened tension line 150 which is attributable to the “User adjusted force”. For the elastic positioning web, the remainder of the force is contributed by the spring force of the positioning web 112. The headgear 110 is removed by unlocking the controller 140 to release the remaining tension in the tension line 150 and then the remainder of the doffing process is the same as described above in respect of FIG. 5.

An alternative adjustment option to provide a feeling of a more secure fitting is shown in FIGS. 10 and 11 where the adjustment occurs after the flow generator is activated. This option is the same as the no-adjustment option (FIGS. 4 and 5) up to the point of activating the flow generator. When the flow generator is activated, the force increases without any change in displacement along the line A on account of the controller 140 being locked. This is denoted in FIG. 10 as the initial phase “Tension line is locked” and is denoted in FIG. 11 as segment ‘A’. However, once the force is in the operating envelope and the user has a feel for the headgear 110, they unlock the controller 140 and tighten the tension line 150 which reduces the displacement (denoted in FIG. 10 by the “Tightened” phase) and increases the force (denoted as “Unlock and tighten” in FIG. 11). Once tightened, the nominal force N is reached and the controller 140 is relocked to fix the displacement of the tension line 150 and positioning web 112 (denoted by the phase “Tension line is locked” and “Pressure on” in FIG. 10). Any variations in the force (such as ventilation gas pressure variation or tube pull force) will then be along the line denoted as “Force-Tension line locked”. When the treatment has ended and the flow generator is deactivated (denoted as the phase “Pressure off” in FIG. 10), the tension force decreases along the segment B of the Force-Tension line locked in FIG. 11. This leaves a small force contribution from tension in the tightened tension line 150. For the elastic positioning web, the remainder of the force is contributed by the spring force of the positioning web 112. This point is denoted in FIG. 11 as the “Unlock point of Tension line”. The headgear 110 is removed by unlocking the controller 140 to release the remaining tension in the tension line 150 (on account of user adjusted tightening). The release of the tension is denoted by arrows R which show a force reduction and a commensurate increase in displacement to the “Lock point of tension line”. At this point, the positioning web 112 is resting on the user's head. The remainder of the doffing process is the same as described above in respect of FIG. 5.

### Headgear Embodiments

Various headgear embodiments will now be described based on the notional division of the positioning web 112 into five regions as shown in FIGS. 2 and 3, namely the first region 102 is at a front lower part of the positioning web, a second region 104 is at a rear lower part, the third region 106 is at a rear upper part above the second region 104 and the fourth region 108 is at a front upper part above the first region 102. In some embodiments, the positioning web 112 includes the fifth region 109.

While the fifth region 109 is shown as part of the positioning web 112 in FIGS. 1 to 3, 15A and 26, the fifth region 109 may be formed as a separate component which is attachable in other examples. However, it should be appreciated that the fifth region 109 is an optional part of the positioning web 112.

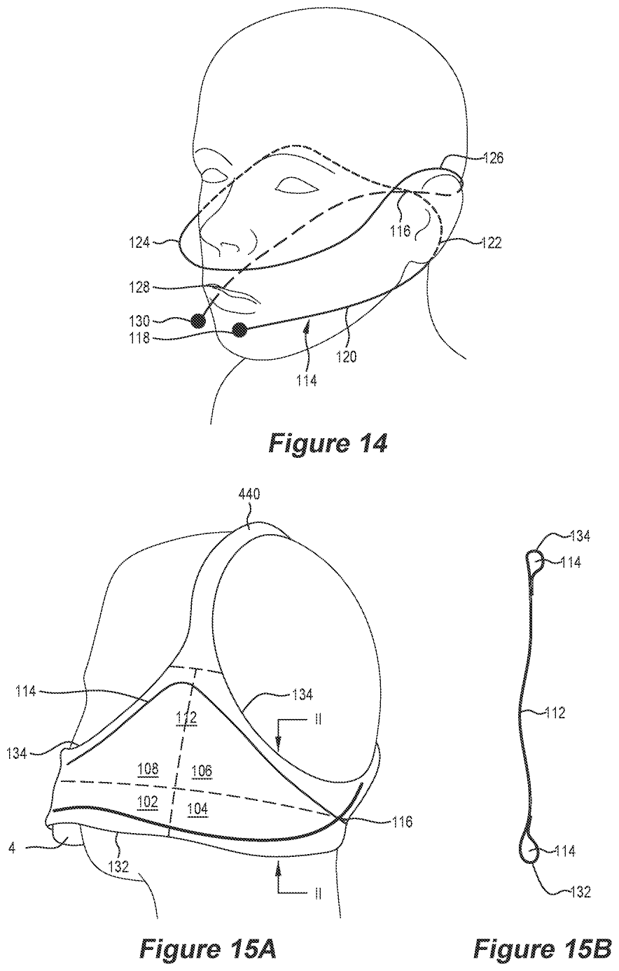

Headgear 100 according to one embodiment is shown in FIGS. 14, 15A and 15B.

In this embodiment, the pathway 114 is formed integrally with the positioning web 112 as part of the process for forming the positioning web 112. In this embodiment, a first pathway portion comprising the first, second and third sections 120, 122 and 124 commences at a first end 118 and extends along the first side in the first and second regions 102 and 104 and then extends along the second side in the third and fourth regions. A second pathway portion comprising fourth and fifth sections 126 and 128 commences at a second end 130 and extends along the second side in the first and second regions 102 and 104 and then extends along the first side in the third and fourth regions 106 and 108. The first and second pathway portions are portions of a single pathway that is continuous across the fourth region 108. Accordingly, the pathway 114 circumscribes the head twice and commences and terminates in the first region 102 at a location that coincides with the location of a respiratory interface 4. As seen in FIGS. 14 and 15A, pathway 114 includes an intersection 116 at a rear part of the positioning web 112. Portions of the pathway are shown in broken lines where they are on the far side of the head in FIG. 14. Other portions of the pathway are shown in solid lines to denote those portions of the pathway that are on the near side of the head. Additionally, the different portions of the pathway on the far side of the head are shown in longer and shorter dashed-lines to differentiate the pathway portions.

Having regard to the headgear 100 when fitted to the user, the path of the pathway 114 can be described as follows. From the first end 118, the first section 120 of the pathway 114 extends along a lower rim 132 of the positioning web 112 and underneath a location coinciding with a first ear of the user. The first section 120 transitions into a second section 122 which traces around the rear of the user's head in a generally diagonal direction so that the pathway 114 extends from the lower rim 132 to an upper rim 134 of the positioning web 112 above a location coinciding with the user's second ear. From that point, a third section 124 of the pathway 114 extends across the user's face from locations coinciding with the user's respective cheek bones and transverses the respiratory interface 4 at a location that coincides with an upper lip region of the user. The third section 124 continues along the upper rim 134 above a location coinciding with the user's first ear and then transitions into a fourth section 126 which traces around the rear of the user's head in a generally diagonal direction from the upper rim 143 to the lower rim 132 below a location coinciding with the user's second ear. In doing so, the fourth section 126 and intersects with the second section 122 at an intersection 116 at the rear of the user's head. The pathway 114 then transitions into a fifth section 128 that extends along the lower rim 132 to the second end 130.

The tension line 150 passes through the first and second ends 118 and 130 and is coupled to the controller 140.

The positioning web 112 shown in FIG. 15A is formed by knitting and it is the knit filaments and the weave which contributes to the elastic properties of the positioning web 112. The elastic properties of the positioning web 112 are selected so that it is elastic in that it has some tendency to maintain its shape, including a tendency to maintain the spacing of the pathway portions about the user's head. Maintaining the spacing is important for two reasons. One reason is that the shape of the band, more specifically the wide band shape about the user's head, assists to distribute pressure applied by the positioning web 112 to the user's head due to tension forces running through positioning web 112. This is important because it contributes to the user's comfort when wearing the headgear 100. The second reason is that the locations of the pathways 114 affects the tension in a tension line 150 located in the pathway 114 and, therefore, the force with which the respiratory interface 4 is held on the user's face. This is a result of the controller 140 having a locked position in which the deployed length of tension line 150 is a set length based on the locations of the pathway 114 when the controller 140 was locked. Movement of the positioning web about the user's head may result in contraction of the positioning web 112 and, therefore, shortening of the pathway 114 such that the length of tension line 150 required to maintain the same tension applied to the respiratory interface 4 is also shorter. The result is that the tension in the tension line 150 decreases and may even result in the tension line 150 being slack. In the absence of adjusting the controller 140 to shorten the tension line 150 length, the tension line 150 may not fully counteract the blow-off force and any tube pull so that there is gas leakage from the interface 4 which may result in the treatment being less effective or being ineffective.

The knitting pattern may provide anisotropic elasticity in the positioning web 112. This may involve the positioning web 112 being more elastic in the height direction than in its length direction to facilitate expansion and contraction for donning and doffing and to assist with maintaining the spacing between pathway portions about the positioning web. The knitting material may also have properties that contribute to anisotropic elasticity of the positioning web. For example, the elasticity of the knitting material may vary along its length. With an appropriate knitting pattern, more elastic portions of the knitting material may generally be aligned in one direction and the less elastic portions may generally be aligned in another direction.

The construction method is not limited to knitting, so the positioning web 112 may be formed by any suitable alternative methods, including weaving, extrusion moulding or injection moulding. These methods are indicative of optional construction methods. It follows that they, or any suitable alternative methods, may be applied to any of the embodiments disclosed here.

Regardless of the construction method, the material from which the positioning web 112 is formed must be biocompatible. It may also be non-allergenic, breathable and may be natural or synthetic material or a blend of both.

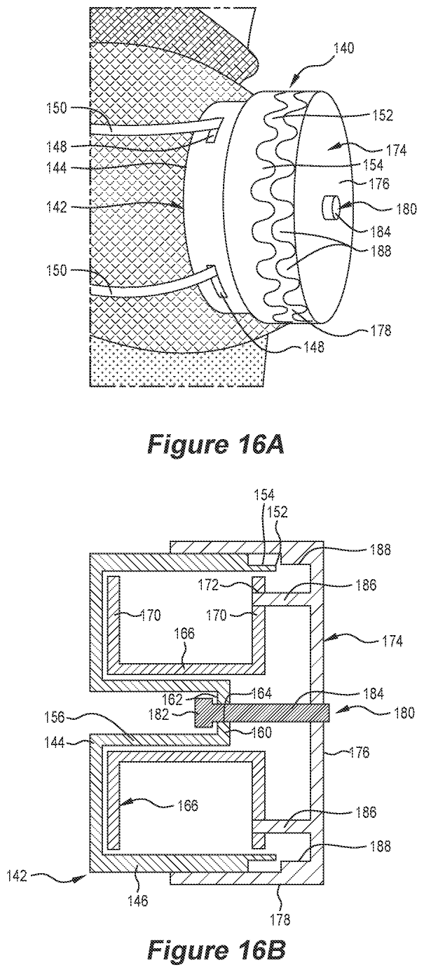

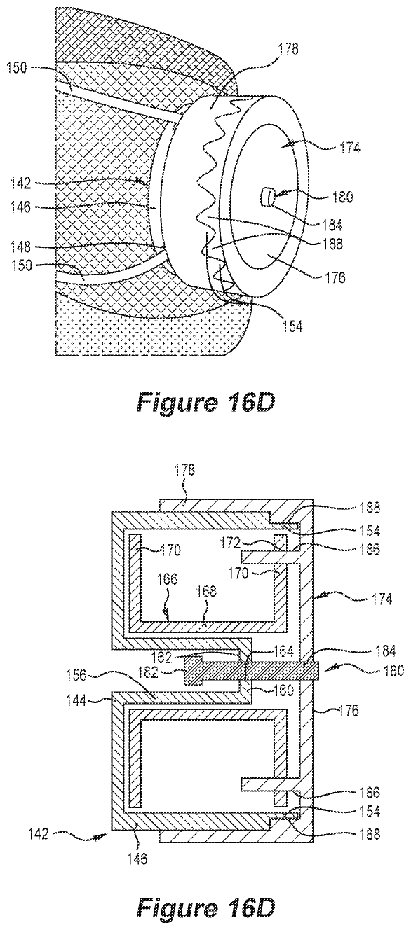

The controller 140 in this embodiment is shown in more detail in FIGS. 16A to 16D. In this example, the controller 140 is incorporated into the respiratory interface 4 and includes a lock that fixes the deployed length of the tension line 150. It will be appreciated, however, that other embodiments may involve the controller 140 being attached to the respiratory interface 4.

While the following description of the controller 140 is in the context of the headgear 100, it will be appreciated that the same controller may be used in other embodiments described below.

In this embodiment, the controller 140 (FIG. 16B) comprises a housing 142 which includes a base 144 and an upstanding and annular wall 146 which projects perpendicularly from the base 142. The annular wall 146 includes side openings 148 adjacent to the base 142 and through which a tension line, in the form of a tension line 150, passes from the controller 140 to the pathway 114. The annular wall 146 also includes an upper rim 152 which is formed as a series of teeth 154 disposed circumferentially about the rim 152 and projecting axially of the annular wall 146. The housing 142 further comprises a central annular boss 156 having, at an end 158 remote from of the base 144, a radially inwardly projecting in flange 160 which defines a shoulder 162 and a central opening 164 in the end of the boss 156.

A reel, in the form of a spool 166, having a hollow cylindrical body 168, is mounted on the annular boss 156 such that it is able to freely rotate on the boss 156. The spool 166 includes radially projecting end flanges 170 at each end of the body 168 such that rotation of the spool 166 allows the tension line 150 to wind onto or unwind from the cylindrical body 168 so that the tension line 150 enters or exits via the side openings 148 in the housing. The annular flange 170 remote from the base includes four openings 172 which are equally spaced circumferentially about an outer annular region of the flange. Each opening 172 has and elongate shape which may be straight or maybe curved to form a segment of a circle.

The controller 140 further includes a dial, in the form of a covering cap 174, having a circular outwardly-facing end face 176 and an annual grip wall 178 extending perpendicularly from the perimeter of the end face 176. An inner side of the annular grip wall has a radius which is slightly greater than the radius of an outer surface of the annular wall 146 of the housing 142. Accordingly, the cap 174 sits over the housing 142 such that the grip wall 178 extends axially to a point slightly short of the side openings 148 in the side wall so as to avoid interference between the grip wall 178 and the tension line 150. The grip wall 178 is retained in that position by an axial displacement peg 180 having an enlarged head 182 that is located within the boss 156 and having an axle 184 extending from the head 182 and which is fixed to the centre of the end face 176 so that the cap 174 is able to freely rotate about the axle 184. The axle 184 as a length which enables the cap 174 to move axially relative to the housing 142.

The cap 184 further includes four drive tabs 186 located on the end face 176 at a position which aligns them with the openings 172 on the outer flange 170. The drive tabs 186 are directed inwardly toward the spool 166 and have a profile shape which corresponds to the shape of the openings 172 in the outer flange 170. Furthermore, the drive tabs 186 have a length which allows them to project into the openings when the cap 174 is axially extended relative to the housing 142 and also when the cap 174 is axially retracted relative to the housing 174, so that the cap 174 is rotationally linked with the spool 166 such that rotation of the cap 174 causes winding of the spool 166.

An inside wall of the annular grip wall 178 includes a series of teeth 188 which are correspondingly shaped to the teeth 154 which form the rim 152 of the housing 142. The profile of the teeth 188 extend radially inwardly from the inner side of the grip wall 178 to align axially with the teeth 154 forming the rim 152. The axial movement of the cap 184 relative to the housing 142 enables the teeth 188 of the cap 174 to move into and out of engagement with the teeth 154 of the housing 142. The teeth 188 and the teeth 154 comprise formations which are engageable to prevent rotation of the cap 174 and are disengageable to permit rotation of the cap 174.

When the cap 178 is axially retracted (FIGS. 16C and 16D), the teeth 156 forming the rim 152 interact with the teeth of the cap such that the cap 178 is rotatably locked relative to the housing 142 and the spool 166 is also rotatably locked due to the drive tabs 186 of the cap 174 being located in the openings 172 of the outer flange 170. Axial extension of the cap 178 relative to the housing 142 disengages the teeth 156, 188 to allow the cap 174 to rotate freely. It follows that the controller 140 enables selective switching between the operative configuration and the donning and/or doffing configuration. Furthermore, rotation of the cap 174 causes a corresponding rotation of spool 166 via the drive tabs 186 so that the tension line 150 is retracted on to the spool 166 or released from the spool 166 depending upon the direction of rotation of the cap 178. In other words, the controller 140 is operable to control the length of tension line deployed to the or each pathway. In this way, the controller 140 is operable to control fit of the headgear in the operative configuration by adjustment of the length of tension line deployed to the or each pathway.

The controller 140 may further include a coil spring fixed between the spool 166 and the housing 142 which operates to retract the tension line 150 into the accumulator 142. The spring constant of the coil spring is selected to assist with taking up slack in the tension line 150 as the positioning web 112 contracts. The coil spring, therefore, contributes to the sizing of the positioning web 112, but does not act to resist blow-off forces because, when the cap 178 is locked to the housing 142, tension in the tension line 150 passes to the spool 166 and rotation of the spool 166 is resisted by the interlocked teeth 156, 188 of the controller 140.

While the teeth 156, 188 are shown in FIGS. 16A and 16C as a series of generally sinusoidally-shaped ridges and valleys, it will be appreciated that teeth having alternative shapes may be adopted provided that they are suitable for interlocking to prevent rotation of the cap 178 relative to the housing 152.

The ends of the tension line 150 are fastened to the spool 166 of the controller 140 which can be operated by the user to adjust the in-use length of tension line outside the controller 140, i.e. the length of the tension line that resides in the pathway 114. This is important for donning and doffing the headgear 110. For example, the resting state of the positioning web 112 will have a length that is slightly less than the circumference of the user's head so that the elastic properties of the headgear 110 cause it to contract snugly about the user's head. In this state, some tension line 150 is contained in the pathway 114 and the remainder of the tension line 150 is spooled in the controller 140. As part of donning the head gear 110, the positioning web 112 is expanded such that is has a length which exceeds the circumference of the user's head to allow the headgear 100 to be placed on the user's head to fit over the ears. Expansion of the positioning web 112 increases the length of the pathway 114 and, therefore, some of tension line 150 that is spooled on the controller 140 needs to be transferred into the pathway 114. Accordingly, the controller 140 is unlocked so that some tension line 150 is unspooled from the controller 140 and enters the pathway 114 to accommodate the increase in length of the positioning web 112.

Other configurations of controller 140 may be adopted in place of the controller 140 described above provided that they allow adjustment of the tension line 150 length in the pathway 114. It follows that the headgear described here are not limited to the form of controller 140 described above. For example, the controller 140 which operates with the headgear 100 shown in FIG. 1 includes two side openings 148 (FIGS. 16A and 16C) through which the tension line 150 enters after exiting the end points 118 and 130 of the pathway 114. Both ends of the tension line 150 are fixed to the controller 140 such that turning of the cap 184 adjusts the length of the tension line 150 in the pathway 114. However, it will be appreciated that only one end of the tension line 150 may be fixed to the controller 140 with the other end of the tension line 150 fixed to the interface 4, for example. In that case, the controller 140 adjusts the length of the tension line 150 in the pathway 114 in the same way, but the displacement of the tension line 150 occurs only at one end. Alternative headgear embodiments described below may include two separate tension lines 150 which are each linked to the same controller 140 such that the controller 140 adjusts the length of both tension lines 150 simultaneously. In this case, both ends of both tension lines 150 may be fixed to the controller 140 or only one end of each tension line 150 may be fixed to the controller 140.

In another example, the controller 140 may include a friction resistor to control the deployed length of the tension line. The friction resistor may permit adjustment of the deployed length when tension in the tension line 150 overcomes a threshold friction applied by the friction resistor. The tension for overcoming the threshold friction is set exceeds the tension for counteracting the blow-off and tube-pull forces. In the above described embodiment, the friction resistor may comprise the spool 166 frictionally interacting with the housing 142.

In another example, the controller 140 may comprise an anchor point on the interface 4 and a toggle which can be releasably fastened to the anchor point (e.g. by snap-fit connection) and to which toggle the tension line can be attached (e.g. by friction-fit). The attachment allows for the deployed length of the tension line to be set so the toggle can be released from the anchor point to enable donning and doffing and can be re-fastened to the anchor point so the deployed length of tension line doesn't need to be adjusted to enable fitting and removal of the headgear. Optionally, the toggle may also allow adjustment of the deployed length. In another example, the tension line 150 may be releasably attached to the anchor point on the mask 4 for the same effect.

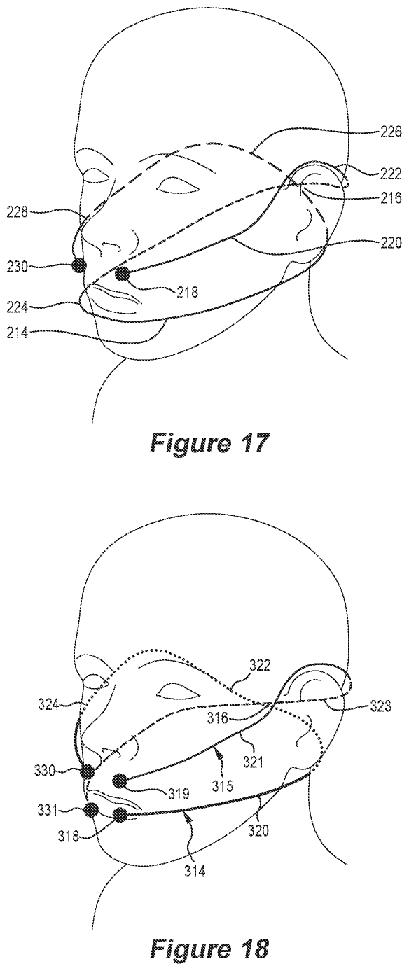

Different embodiments will now be described with reference to FIGS. 17 to 37. As with FIG. 14, portions of the pathway or pathways are shown in broken lines where they are on the far side of the head in FIGS. 17 to 37. Other portions of the pathway or pathways which are shown in solid lines denote those portions of the pathway that are on the near side of the head. Additionally, embodiments having two or more pathways denote the different pathways on the near side of the head with different line weights only in order to distinguish them. The different line weights are not to be construed that the different pathways carry different tension (although they may) and are not to be construed as an order of importance. On the far side of the head, the different pathways are denoted by different broken line styles, e.g. dashed lines and dotted lines.

A variation of the headgear 100 involves an alternative pathway 214 shown in FIG. 17. The form of the positioning web 112 remains the same, but the pathway 214 follows an alternative path. Features in this embodiment that are the same as features in the previous embodiment are denoted by like reference numbers, but preceded with a “2”.

The end points 218, 230 of the pathway 214 coincide with an upper portion of the respiratory interface 4. More specifically, the first end point 218 and the second end point 230 of the pathway 214 are positioned at locations that coincide with respective sides of the upper lip of the user. These locations will be close to or on top of the interface 4. From the first end 218 of the pathway 214, a first section 220 of the pathway 214 extends along an upper rim 134 of the positioning web 112 and above a location coinciding with a first ear of the user, then a second section 222 transitions around the rear of the user's head in a generally diagonal direction so that the pathway 214 extends from the upper rim 134 to a lower rim 132 of the positioning web 112 below a location coinciding with the user's second ear. From that point, a third section 224 of the pathway 214 extends across locations coinciding with the user's lower jaw and transverses the respiratory interface 4 at a location that coincides with an area between the lower lip of the user and their chin. The third section 224 continues along the lower rim 132 below a location coinciding with the user's first ear and then transitions into a fourth section 226 that transitions around the rear of the user's head in a generally diagonal direction so that the pathway 214 extends from the lower rim 132 to the upper rim 134 above a location coinciding with the user's second ear. The pathway 214 then transitions into a fifth section 228 that extends along the upper rim 134 to the second end point 230.

The controller 140 is located at the ends 218, 230 of the pathway 214 so that ends of the tension line 150 disposed in the pathway 214 are coupled to the controller 140 so that it can control the length of tension line 150 that is deployed into the pathway 214.

Having regard to the notional division of the positioning web 112 into regions which are shown in FIGS. 2 and 3, a first pathway portion comprising the first, second and third sections 220, 222 and 224 commences at a first end 218 and extends along the first side in the fourth and third regions 108, 106 and then extends along the second side in the second and first regions 104, 102. A second pathway portion comprising fourth and fifth sections 126 and 128 commences at a second end 130 and extends along the second side in the first and second regions 102 and 104 and then extends along the first side in the third and fourth regions 106, 108. The first and second pathway portions are portions of a single pathway that is continuous across the fourth region 108. Accordingly, the pathway 214 circumscribes the head twice and commences and terminates in the fourth region 108 at a location that coincides with the location of a respiratory interface 4. As seen in FIG. 17, pathway 214 includes an intersection 216 at a rear part of the positioning web 212.

A variation of the embodiment shown in FIGS. 14 and 17 is shown in FIG. 18 in which positioning web 112 is the same as in FIG. 15A, but it has two tension lines which are disposed in respective pathways 314 and 315. The four ends of the two tension lines connect to the controller 140 at the interface 4. Features in this embodiment which are the same as features in preceding embodiments are denoted by like reference numerals, but proceeded with the number “3”.

In this embodiment, a first pathway 314 has a first end point 318 adjacent to a lower portion of the interface 4 and from which extends a first section 320 of the pathway 314 along a lower rim 132 of the positioning web 112. The first section 320 extends to below a location coinciding with a first ear of the user and then transitions into a second section 322 which transitions from the lower rim 132 to the upper rim 134 as it extends about a portion of the positioning web located on the rear of the user's head. The second section 322 extends above a location coinciding with the user's second ear where the pathway 314 transitions into a third section 324 which extends along the upper rim 134 to a second end point 330 coinciding with an upper portion of the interface 4. The second pathway 315 does the reverse of the first pathway 314 in that it commences at a first end point 319 which coincides with an upper portion of the interface 4 above the first end point 318 of the first pathway and has a first section 321 extending from the first end point 319 along the upper rim 134 of the positioning web to a location above a location coinciding with the user's first ear. The pathway 315 then transitions into a second section 323 which transitions across the positioning web 112 from the upper rim 134 to the lower rim 132 along a portion of the positioning web 112 that coincides with the rear of the user's head. In making that transition, the pathway 315 intersects the pathway 314 so that tension lines in the respective pathways 314 and 315 cross-over each other at the intersection 316. The pathway 315 extends below the user's second ear and transitions into a third section 325 which extends along the lower rim 332 to the second end point 331 that coincides with a lower portion of the mask of below the second end point 330 of the first pathway 314. The controller 140 is located at the ends of the pathways 314 and 315 so that ends of tension lines in the pathways 314 and 315 are coupled to the controller 140 so that the controller 140 can control the length of tension line 150 that is deployed into the pathways 314 and 315.

Having regard to the notional division of the positioning web 112 into regions which are shown in FIGS. 2 and 3, a first pathway 314 comprising the first and second sections 320, 322 commences at a first end 318 and extends along the first side in the first and second regions 102, 104 and then extends along the second side in the third and fourth regions 106, 108. A second pathway 315 comprising first and second sections 321 and 323 commences at a second end 319 and extends along the first side in the fourth and third regions 108, 106 and then extends along the first side in the second and first regions 104, 102. The first and second pathways 314 and 315 are separate pathways. As seen in FIG. 18, the first and second pathways 314 and 315 intersect at a rear part of the positioning web 312 at intersection 316.

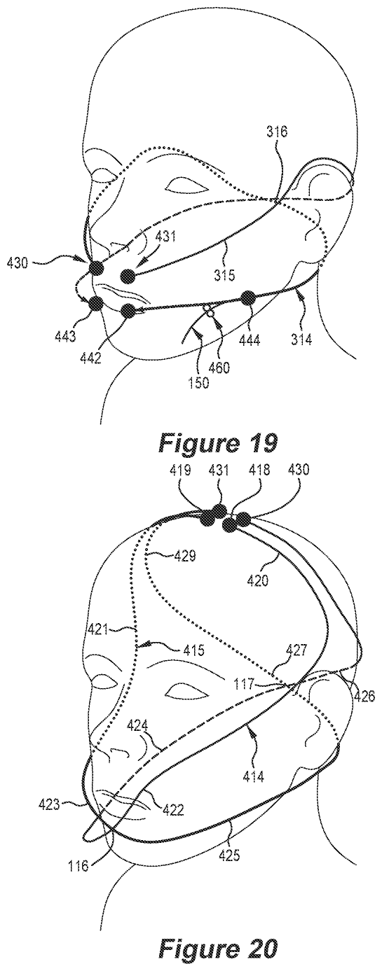

A variation on the embodiment shown in FIG. 18 is shown in FIG. 19 and involves the first and second pathways 314 and 315 each having one end 444, 445 that is remote from the interface 4 and another end 430 and 431 that is located to coincide with the interface 4. Tension lines 150 are disposable in the pathways 314 and 315 and each is linked to a separate controller 140, which is in the form of a break-fit connection in this embodiment. A break-fit connection has a locked configuration to which it is biased, but when a sufficient force is applied the connection moves to an unlocked configuration. In the unlocked configuration, the positioning web 112 is able to freely expand whereas in the locked configuration, the positioning web 112 is prevented from expanding. Because the mechanism is biased towards its locked configuration, it returns to this configuration when the applied force is removed or sufficiently reduced. The break-fit connection prevents inadvertent expansion of the positioning web 112 under relatively low forces (e.g. blow off and hose pull). Examples of two magnetic break-fit connections are described in PCT/NZ2013/000139 in the name of Fisher & Paykel Healthcare Limited in connection with FIGS. 9 and 10.

In another example of a break-fit connection, the controller 140 comprises an anchor point 442 on the interface 4 or the positioning web 112 and a tab 460 fastened to the tension line 150 (shown projecting from the end 444 of the pathway 314 in FIG. 19). The tab 460 can be shifted along the length of the tension line 150 to adjust the length of tension line 150 that is available to pass into the pathway 314 or 315. The tab 460 is configured to be releasably fixed to the anchor point 442. In this embodiment, the tab 460 has a FIG. 8 shape about which the tension line is wrapped to fix the position of the tab 460 along the tension line 150. Typically, the anchor point is at the front of the headgear 410 to make it easier for a user to fix and release the tab 460, or other break-fit connection. The anchor point 442 has a formation that co-operates with a fixing tab 460 to which the tension line is connected and which releasably fixes the tension line 150 to the anchor point 442. The tension line 150 may be connected to the fixing tab 460 permanently (for example, by welding), mechanically or by friction fit. In the case of the latter two connection forms, the location of the tab 460 along the length of the tension line 150 is adjustable.

In this circumstance, the headgear 112 is donned in the normal manner as described above for the embodiment associated with FIG. 1 in that the controller 140 is unlocked so the tension line 150 is able to slide freely through the pathway 314. In other words, the tab 460 is fixed to the anchor but the tab 460 is not fixed to the tension line 150. Once fitted, the user fixes the tension line 150 to the tab 460 by removing slack in the tension line 150 so the length of tension line 150 between the anchor point 442 and the end 430 through the pathway 314 is roughly equally to the length of the pathway 314 plus the distance between the end 444 and the anchor point 442. This step fixes the length of tension line 150 in the pathway 314. That same action is performed with the tab 460 and tension line associated with the pathway 315.

When the headgear is doffed, instead of unlocking the controller 140, i.e. releasing the tension line 150 from the tab 460, the user can release the tab 460 from the anchor point 442 to enable the tension line 150 to flow freely through the pathway 314. The positioning web 112 can then be expanded to remove the headgear 100. However, when the headgear 100 is donned again, the user can simply re-connect the tab 460 to the anchor point 442 to fix the length of tension 150 without having to adjust its deployed length. It will be appreciated that the break-fit connection avoids the need for adjustment of the headgear 100 via the controller 140 each time the headgear is donned or doffed. The same applies to the pathway 315 in FIG. 19 when the tension line 150 located in the pathway 315 has a fixing tab 460 which is fastenable to the anchor point 443

A variation of the embodiment shown in FIGS. 14 and 17 is shown in FIG. 20 in which the positioning web 112 is the same as in FIG. 15A, but the controller 140 is located in the fifth region 109 (see FIGS. 2 and 3). In this embodiment, the headgear 110 has two pathways 414 and 415 with respective end points 418, 430 and 419, 431 adjacent the controller 140 in the fifth region 109.

In this embodiment, a first pathway 414 (comprising sections 420, 422, 424 and 426) commences at the fifth region 109 of the positioning web 112 and then extends forwardly of the fifth region in the fourth region 108 on the first side. The first pathway 414 then transitions into the first region 102 and extends rearwardly in the first and second regions 102, 104 on the second side. The first pathway 414 then transitions into the third region 106 and extends along the first side back to the fifth region 109. The second pathway 415 (comprising sections 421, 423, 425 and 427) commences at the fifth region 109 of the positioning web 112 and then extends forwardly of the fifth region 109 in the fourth region 108 on the second side. The second pathway 415 then transitions into the first region 102 and extends rearwardly in the first and second regions 102, 104 on the first side. The second pathway then transitions into the third region 106 and extends along the second side back to the fifth region 109.

More specifically, the pathway 414 commences at the first end point 418 in the fifth region 109 and the first section 420 extends along the fifth region and into the upper rim 134 above a first ear of the user. The first section 420 continues along the upper rim 134 toward the interface 4 and then transitions into the second section 422 which extends diagonally across the interface 4 from the upper rim 134 to the lower rim 132 where the pathway 414 transitions into a third section 424 that extends along the lower rim 132 of the headgear 110 and underneath a second ear of the user. The pathway 414 then transitions into the fourth section 426 which transitions from the lower rim 132 to the upper rim 134 across the rear of the user's head in a generally diagonal direction. The fourth section 426 extends from the upper rim 134 into the fifth region and terminates at the second end point 430 which is adjacent to the first end point 418. The second pathway 415 originates at the first end point 419 on an opposite side of the controller 140 to the first end point 418 of the first pathway 414 and extends, as the first section 421, away from the first pathway 414 on the opposite side of the fifth region 109. The first section 421 extends into the upper rim 134 above a second ear of the user. The first section 421 then continues along the upper rim 134 toward the interface 4 and then transitions into a second section 423 which extends diagonally across the interface 4 from the upper rim 134 to the lower rim 132 and intersects the first pathway 414 at an intersection 116 coinciding with the interface 4. The second pathway 415 then transitions into a third section 425 that extends along the lower rim 132 of the headgear 110 and underneath the first ear of the user. The pathway 415 then transitions into a fourth section 427 which transitions from the lower rim 132 to the upper rim 134 across the rear of the user's head in a generally diagonal direction and intersects the first pathway 414 again at an intersection 117. A fifth section 429 extends from the upper rim 134 into the fifth region 109 and terminates at the second end point 431 which is adjacent to the first end point 419 and opposite the second end point 430 of the first pathway 414.

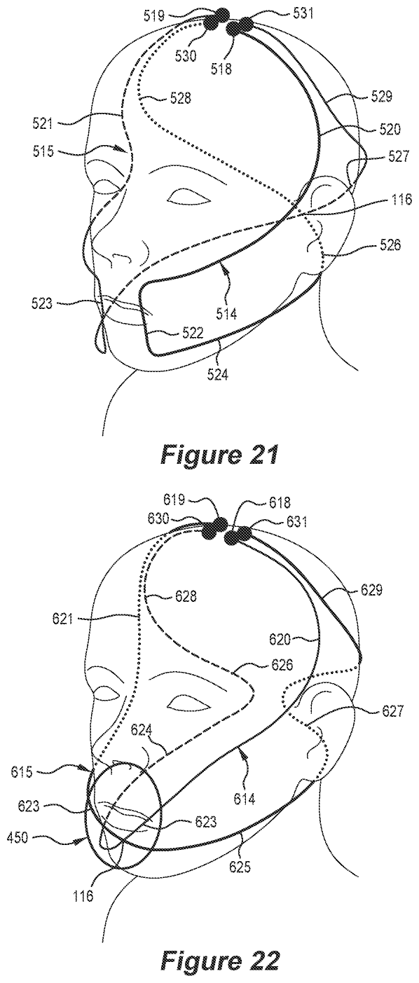

A variation of the embodiment in FIG. 20 is shown in FIG. 21 where the pathways do not intersect at a location coinciding with the interface 4.

According to this embodiment, the first pathway 514 (comprising sections 520, 522, 524, 526 and 528) commences at the fifth region 109 on the first side of the positioning web 109 at end point 518, extends along the fifth region 109, transitions into the fourth region 108 and then into the first region 102 forwardly of the fifth region 109. The first pathway 514 then extends rearwardly on the first side along the first and second regions 102, 104, then transitions into the third region 106 and extends into the fifth region 109 on the second side. The second pathway 515 (comprising sections 521, 523, 525, 527 and 529) commences at the fifth region 109 on the second side and then transitions into the fourth region 108 and then the first region 102 on the second side forwardly of the fifth region 109. The second pathway 515 then extends rearward on the second side along the first and second regions 102, 104 and then transitions into the third region 106 and extends into the fifth region on the first side.

More specifically, the pathway 514 has a first end point 518 in the fifth region 109 and a first section 520 extending along the fifth region 109 on the first side and into the upper rim 134 above a first ear of the user. The first section 520 continues along the upper rim 134 toward the interface 4 and then transitions into a second section 522 which extends downwardly from the upper rim 134 to the lower rim 132 without extending diagonally across the interface 4 as in the embodiment shown in FIG. 20. At the lower rim 132, the pathway 514 transitions into a third section 524 that returns along the positioning web 112 along the lower rim 132 of the headgear 110 on the first side and underneath the first ear of the user. From there, the pathway 514 transitions into a fourth section 526 which transitions from the lower rim 132 to the upper rim 134 across the rear of the user's head in a generally diagonal direction and above the user's second ear. A fifth section 528 extends from the upper rim 134 into the fifth region 109 and terminates at the second end point 530 which is opposite the first end point 518. The second pathway 515 originates in the fifth region 109 at a first end point 519 on an opposite side of the controller 140 to the first end point 518 of the first pathway 514. The second pathway 515 extends, in a first section 521, away from the first pathway 514 along on the opposite side of the fifth region. The first section 521 extends into the upper rim 134 above a second ear of the user. The first section 521 then continues along the upper rim 134 toward the interface 4 and then transitions into a second section 523 which extends downwardly from the upper rim 134 to the lower rim 132 without extending diagonally across the interface 4 as in the embodiment show in FIG. 20. At the lower rim 132, the pathway 515 transitions into a third section 525 that returns along the positioning web 112 on the second side along the lower rim 132 of the headgear 110 and underneath the second ear of the user. The pathway 515 then transitions into a fourth section 527 which transitions from the lower rim 132 to the upper rim 134 across the rear of the user's head in a generally diagonal direction and intersects the first pathway 514 at an intersection 116. A fifth section 528 extends from the upper rim 134 into the fifth region 109 and terminates at the second end point 531 which is opposite to the first end point 519 and adjacent the first end point 518 of the first pathway 514.

The controller 140 is located between all of the end points 518, 519, 530 and 531. It may be linked to both ends of each tension line 150 or may be linked to one end of each tension line with the other end fixed to the positioning web 112 or to the controller 140.

A variation of the embodiment shown in FIG. 21 is shown in FIG. 22 in which pathways 614 and 615 intersect at a location coinciding with the interface 4 but do not intersect at the rear of the user's head. Specifically, the headgear includes a respiratory interface region 450 (see FIG. 22) and the first pathway 614 (comprising sections 620, 622, 624, 626 and 628) commences at the fifth region 109 on the first side of the positioning web, transitions into the fourth region 108 on the first side and then transitions into the first region 102 via the interface region 450. The first pathway 614 then extends rearwardly along the first and second regions 102, 104 on the second side, then transitions into the third region 106 and extends into the fifth region on the second side. Additionally, the second pathway 615 (comprising sections 621, 623, 625, 627 and 629) commences at the fifth region 109 on the second side, transitions into the fourth region 108 on the second side and then transitions into the first region 102 forwardly of the fifth region 109. The second pathway 615 then extends rearwardly on the first side along the first and second regions 102, 104, then transitions into the third region 106 and extends into the fifth region 109 on the first side.

More specifically, the first pathway 614 has a first end point 618 in the fifth region 109 and the first section 620 extends along the fifth region 109 and into the upper rim 134 above a first ear of the user. The first section 620 continues along the upper rim 134 toward the interface 4 and then transitions into a second section 622 which extends diagonally across the interface 4 from the upper rim 134 to the lower rim 132 where the pathway 614 transitions into a third section 624 that extends along the lower rim 132 of the headgear 110 and underneath a second ear of the user. The pathway 614 then transitions into a fourth section 626 which transitions from the lower rim 132 to the upper rim 134 at the rear of the user's head without crossing from one side of the user's head to the other at the rear. In other words, the pathway 614 remains on the one side of the head and takes the form of a sideways V (i.e. “>” shape), albeit curved, in the transition between the lower rim 132 and the upper rim 134. A fifth section 628 extends from the upper rim 134 into the fifth region 109 and terminates at the second end point 630 which is opposite to the first end point 618. The controller 140 is located adjacent to the end points 618 and 630 in the fifth region 109.

The second pathway 615 originates at a first end point 619 on an opposite side of the controller 140 to the first end point 618 of the first pathway 614 and a first section 621 extends away from the first pathway 614 along on the opposite side of the fifth region 109. The first section 621 extends into the upper rim 134 above a second ear of the user. The first section 621 then continues along the upper rim 134 toward the interface 4 and then transitions into a second section 623 which extends diagonally across the interface 4 from the upper rim 134 to the lower rim 132 and intersects the first pathway 614 at an intersection 116 coinciding with the interface 4. The second pathway 615 then transitions into a third section 625 that extends along the lower rim 132 of the headgear 110 and underneath the first ear of the user. The pathway 615 then transitions into a fourth section 627 which transitions from the lower rim 132 to the upper rim 134 at the rear of the user's head without crossing from one side of the user's head to the other. In other words, the pathway 615 remains on the one side of the head and takes the form of a sideways V (i.e. “>” shape), albeit curved, in the transition between the lower rim 132 and the upper rim 134. A fifth section 629 extends from the upper rim 134 into the fifth region 109 and terminates at a second end point 631 which is opposite to the first end point 619 and adjacent the first end point 618 of the first pathway 614.

The controller 140 is located in the fifth region 109 between all of the end points 618, 619, 630 and 631 and performs the same function of adjusting the length of the tension lines contained in the pathways 614 and 615. However, as with the previous embodiment, the controller 140 may be linked to both ends of each tension line 150 or may be linked to one end of each tension line with the other end fixed to the positioning web 112 or to the controller 140.

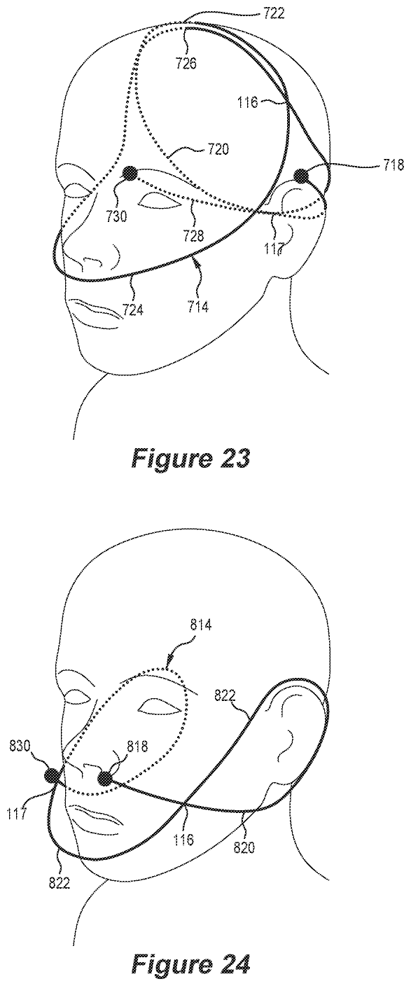

An embodiment which utilises the same positioning web 112 as shown in FIG. 15A and an alternative pathway path is shown in FIG. 23. In this embodiment, a first portion (comprising sections 720, 722 and 724) of the pathway 714 extends from the third region 106 on the first side, across the rear of the user's head and into the fifth region 109 on the second side. The pathway 714 then extends across the fifth region 109 to the fourth region 108 on the first side and forwardly along the fourth region. A second portion (comprising sections 726 and 728) of the pathway 714 extends from the third region 106 on the second side, across the rear of the user's head and into the fifth region 109 on the first side. The pathway 714 then extends across the fifth region 109 to the fourth region 108 on the second side and forwardly along the fourth region 108.

Ends 718 and 730 of the pathway 714 are located adjacent to the fifth region 109 on the first side and on the second side respectively. In this embodiment, the pathway 714 is a single pathway which is continuous across the fourth region 108.

In one variation, the pathway 714 may extend from the third region 106 to the second region 104 about the rear of the user's head and may return to the third region 106 before transitioning into the fifth region 109. The first and second pathway portions may transition into the first region from the fourth region.

The route of the pathway can be described in the following terms. The pathway 714 commences at a first end point 718 above the user's first ear and extends across the rear of the user's head in a first section 720 which extends downwardly from above the first ear to the lower rim 132 and then back up to the upper rim 134, from there, the pathway 714 transitions into a second section 722 which extends through the fifth region 109 across the top of the user's head from the second ear back toward the first ear. The pathway 714 then transitions into a third section 724 which extends along the upper rim 134 across the user's face from the first ear to the second ear, including extending across the interface 4 at a location that coincides with the upper lip of the user. The pathway 714 then transitions into a fourth section 726 which extends through the fifth region 109 across the top of the user's head and intersects with the second section 722 at an intersection point 116 along the fifth region 109. The pathway 714 then transitions into a fifth section 728 which extends from the fifth region 109 and around the rear of the user's head along a path that reaches the lower rim 132 at the rear of the user's head and then extends back up to the second end point 730 above the user's second ear. The fifth section 728 intersects the first section 720 at the intersection point 117 at the lower rim 132 at the rear of the user's head. The controller 140 may be located at the first end point 718 or the second end point 730 with the tension line 150 fixed to the positioning web adjacent the other end. Alternatively, a controller 140 may be located at each of the end points 718 and 730. In a further alternative, the controller 140 may be located at one of the intersection points 116, 117.

FIG. 23 shows the intersection point 116 in the fifth region 109 occurring above the user's first ear. However, it will be appreciated that the intersection point 116 may occur at any point along the fifth region 109, but typically will occur at the top of the user's head. While this embodiment is described as having a pathway 714 with separate second and fourth sections 722 and 726 extending through the fifth region 109, the fifth region 109 may include a single pathway for the tension line 150 across the top of the user's head and the pathway may split into forward and rearward portions above the user's ears to provide pathway portions that extends toward the front and rear of the user's head.

An alternative pathway for the positioning web shown in FIG. 15A is shown in FIG. 24. A first portion (comprising sections 820 and 822) of a pathway 814 extends rearwardly along the first side from the fourth region 108 to the first region 102. The first portion then extends through the second region 104 and the third region 106 before extending forwardly along the first side into the fourth region 108 and then the first region 102. A second portion (comprising section 824) of the pathway 814 extends rearwardly along the second side from the fourth region 108 to the first region 102. The second portion then extends through the second region 104 and the third region 106 before extending forwardly along the second side into the fourth region 108 and then the first region 102.

In this particular embodiment, a first section 820 of the pathway 814 extends from a first end point 818 at an upper portion of the interface 4 and extends along the positioning web 112 to the lower rim 132 below the user's first ear. The pathway 814 then transitions from the lower rim 132 to the upper rim 134 about the rear of the user's first ear and transitions into a second section 822 which extends from the upper rim 134 above the first ear along the positioning web 112 to the lower rim 132 across the interface 4 which coincides with location approximating the chin of the user. The second section 822 then extends along the positioning web 112 up to the upper rim 134 above the user's second ear. The pathway 814 then transitions into a third section 824 which extends from the upper rim 134, about the rear of the user's second ear to the lower rim 132 below the user's second ear and then back along the positioning web 112 to the upper rim 134 at the interface 4. The pathway 4 terminates at a second end point 830 which is adjacent to the first end point 818 at a location coinciding with the upper lip of the user. This pathway 814 path involves the second section 822 intersecting the first section 820 at a first intersection point 116 and intersecting the third section 824 at a second intersection point 117.

A variation of the embodiment in FIG. 24 involves the pathway 814 being inverted so the first and second end points 818, 830 are located on the interface at a location that coincides with the chin of the user. In this variation, the second section 822 extends across the interface 4 at a location coinciding with the upper lip of the user.

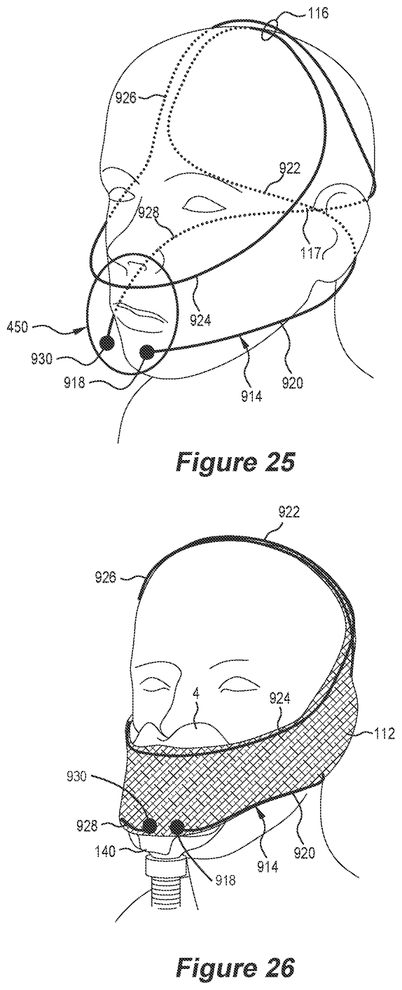

A further embodiment which utilises the same positioning web 112 shown in FIG. 15A, and which is also shown in FIG. 26, has a pathway 914 that is shown in FIGS. 25 and 26.

In this embodiment, the first portion (comprising sections 920, 922 and 924) of the pathway 914 extends rearwardly along the first and second regions 102, 104 on the first side and then into the third region 106 at the rear of the positioning web 112. The first portion then continues along the fifth region 109 from the second side to the first side and returns forwardly along the fourth region 108. The second portion (comprising sections 926 and 928) of the pathway 914 extends along the first and second regions 102, 104 on the second side and the into the third region 106 at the rear of the positioning web 112. The second portion then continues along the fifth region 109 from the second side to the first side and then forwardly along the fourth region 108. The first pathway portion and the second pathway portion are portions of a single pathway which crosses the respiratory interface region 450.

More specifically, the pathway 914 commences at a first end point 918 at a lower portion of the interface 4 and extends in a first section 920 along the lower rim below the user's first ear. The pathway 914 then transitions into a second section 922 which extends diagonally across the rear of the user's head from the lower rim 132 to the upper rim 134 and into the fifth region 109 above the user's second ear. The second section 922 continues along the fifth region 109 across the top of the user's head from the second ear back toward the first ear. The pathway 914 then transitions into a third section 924 which extends along the upper rim 134 which traverses the user's face from the first ear to the second ear, including extending across the interface 4 at a location that coincides with the upper lip of the user. The pathway 914 then transitions into a fourth section 926 which extends along the fifth region 109 across the top of the user's head and intersects with the second section 922 at an intersection point 116 along the fifth region 109. The fourth section 926 continues from the fifth region and diagonally across the positioning web 112 around the rear of the user's head to the lower rim 132 below the user's second ear. The fourth section 926 intersects the second section 922 at an intersection point 117 at the rear of the user's head. The pathway 914 then transitions into a fifth section 928 which follows the lower rim 132 to a lower portion of the interface 4 and terminates at a second end point 930 which is opposite the first end point 918. The controller 140, as with previously described embodiments, is located on the mask between the end points 918 and 930. This enables tension line to be fed into the pathway 914 or retracted from the pathway 914 at the end points 918 and 930 from the controller 140.

Alternatively, each of the end points 918 and 930 may have an associated controller 140. In a further alternative, the controller 140 may be located at one of the intersection points 116, 117.

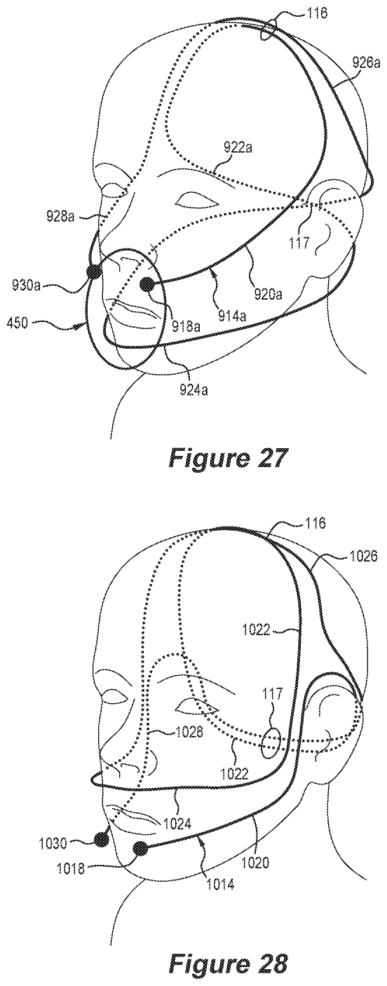

A variation of the embodiment shown in FIGS. 25 and 26 is shown in FIG. 27. A comparison of FIGS. 25 and 27 shows that the pathways 914 and 914a are the same except for the third section 924 in FIG. 25 being replaced by the first and fifth sections 920a and 928a in FIG. 27 and by the first and fifth sections 920 and 928 in FIG. 25 being replaced by the third section 924a in FIG. 27. This means that the third section 924a loops across the lower part of the respiratory interface region 450 and the first and second end points 918a and 930a are positioned at the upper part of the respiratory interface region 450 in the embodiment shown in FIG. 27.

More specifically, the pathway 914a commences at a first end point 918a at an upper portion of the respiratory interface region 450 and extends in a first section 920a along the upper rim 134 to above the user's first ear. The pathway 914a then transitions into a second section 922a which extends along the fifth region 109 across the top of the user's head from the first ear back to the second ear. The pathway 914a then extends diagonally across the rear of the user's head from the upper rim 134 to the lower rim 132 at a position below the user's first ear. The second section 922a then transitions into a third section 924a which extends along the lower rim 134 and which traverses the user's face from the first ear to the second ear, including extending across the respiratory interface region 450 at a location that coincides with the chin of the user. The pathway 914a then transitions into a fourth section 926a which extends diagonally across the positioning web 112 around the rear of the user's head from the lower rim 132 below the user's second ear to above the user's first ear. The fourth section 926a intersects the second section 922a at an intersection point 117 at the rear of the user's head. The fourth section 926a continues along the fifth region 109 across the top of the user's head and intersects with the second section 922a at an intersection point 116 along the fifth region 109. The fourth section 926a then transitions into a fifth section 928a which follows the upper rim 132 from above the user's second ear to the upper portion of the respiratory interface 4 and terminates at a second end point 930a which is opposite the first end point 918a. The controller 140, as with previously described embodiments, is located on the mask between the end points 918a and 930a. This enables tension line to be fed into the pathway 914a or retracted from the pathway 914a at the end points 918a and 930a from the controller 140.

Another embodiment is shown in FIG. 28 which utilises the positioning web 112 shown in FIGS. 2, 3 and 26 with an alternative pathway 1014.

In this embodiment, a first portion (comprising sections 1020, 1022 and 1024) of the pathway 1014 extends rearwardly along the first region 102 on a first side of the positioning web 112 and then transitions through the fourth region 108 and the third region 106 before extending along the second region 104 from the first side to the second side. The first portion then extends into the third region 106 and then along the fifth region 109 from the second side to the first side before then extending forwardly along the fourth region 108 on the first side. A second portion (comprising sections 1026 and 1028) of the pathway 1014 rearwardly long the first region 102 along the second side and then transitions through the fourth region 108 and then the third region 106 before extending along the second region from the second side to the first side. The second portion then extends into the third region 106 and then along the fifth region 109 from the first side to the second side before then extending forwardly along the fourth region 108 on the second side.

More specifically, the pathway 1014 commences at a first end point 1018 at a lower portion of the interface 4 and extends in a first section 1020 along the lower rim 132 to a point just in front of the user's first ear and then extends up and around the first ear and then back down to the lower rim 132 at the rear of the first ear. The pathway 1014 then transitions into a second section 1022 which extends along the lower rim 132 across the rear of the user's head to a position behind the user's second ear and, from there, turns upwardly and extends into the fifth region 109 above the user's second ear. The second section 1022 continues along the fifth region 109 diagonally across the top of the user's head from behind the second ear to in front of the first ear. The pathway 1014 then transitions into a third section 1024 which extends along the upper rim 134 which traverses the user's face from in front of the first ear to in front of the second ear, including extending across the interface 4 at a location that coincides with the upper lip of the user. The pathway 1014 then transitions into a fourth section 1026 which extends through the fifth region 109 diagonally across the top of the user's head to the lower rim 132 behind the user's first ear and then continues along the lower rim 132 to a position behind the user's second ear. At this point the pathway transitions into a fifth section 1028 which extends up and around the second ear and then back down to the lower rim 132 in front of the second ear. From this point, the fifth section extends along the lower rim 132 to a lower portion of the interface 4 and terminates at a second end point 1030 which is opposite the first end point 1018.

The fourth section 1026 intersects the second section 1022 at an intersection point 116 along the fifth region 109. Another intersection of the second section 1022 with the fourth section 1026 occurs in a region behind the first and second ears and along the lower rim 132 at the rear of the head. While this point is shown as intersection 117 in FIG. 28, there is zero spacing or close to zero spacing between the second section 1022 and the fourth section 1026 in that region, so the intersection may be at any point in that region.

In a variation of this embodiment, the second section 1022 and the fourth section 1026 of the pathway 1014 may be combined into a single pathway portion in that region so that a tension line passes through that portion of the pathway 1014 twice.

The controller 140, as with previously described embodiments, is located on the interface 4 between the end points 1018 and 1030. This enables tension line to be fed into the pathway 1014 or retracted from the pathway 1014 at the end points 1018 and 1030 from the controller 140. Alternatively, a controller 140 may be located at each of the end points 1018 and 1030. In a further alternative, the controller 140 may be located at one of the intersection points 116, 117.

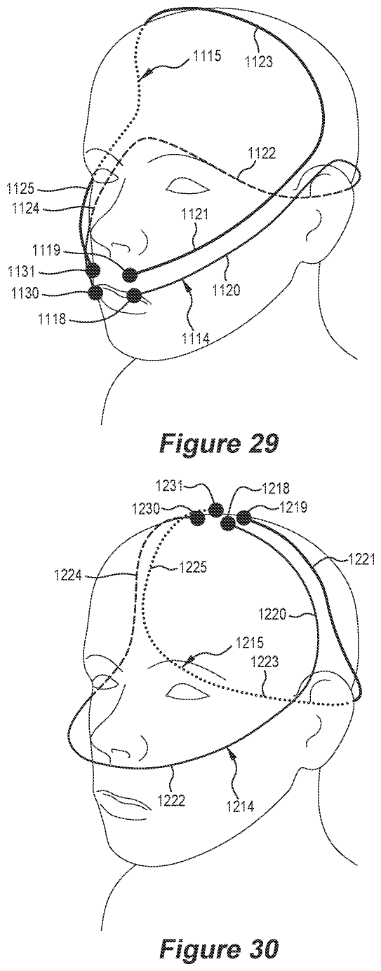

Embodiments in FIGS. 29 to 36 involve two separate pathways which don't intersect with each other, so the tension lines do not cross over. However, each of those embodiments still benefit from the operation of the positioning web distributing the tension force from the tension lines through the positioning web and still operate on the basis that the resting force that the user experiences is the spring force applied by the positioning web 112. They still include one or more controllers 140 which are locked during a treatment so that the instantaneous blow-off force and the tube pull are counteracted by the tension lines 150. Accordingly, these embodiments still have the same advantages that are described in respect of the embodiment shown in FIGS. 1 to 3 with regard to the positioning web 112 and the tension line 150, including the same advantages for donning, doffing and during treatment.

One such embodiment which utilises the same positioning web 112 shown in FIGS. 15A and 26 includes a pathway path as shown in FIG. 29. In this particular embodiment, a first portion (comprising sections 1121, 1123 and 1125) of a pathway 1114 extends rearwardly along the fourth region 108 on the first side. The first portion then extends from the first side to the second side around the rear of the user's head by passing through the third region 106, the second region 104 and then back to the third region 106. From there, the first portion extends forwardly along the fourth region 108 on the second side. A second portion (comprising sections 1120, 1122 and 1124) extends rearwardly along the fourth region 108, then along the fifth region 109 from the first side to the second side and then forwardly along the fourth region 108 on the second side.

More specifically, the positioning web 112 includes two pathway paths. Specifically, a first pathway 1114 includes a first section 1120 which extends from a first end point 1118 along the upper rim 134 to a position above the first ear and then transitions into a second section 1122 which extends diagonally across the positioning web 112 to the lower rim 132 at the rear of the user's head and then extends diagonally upwardly to a position above the second ear. The pathway 1114 then transitions to a third section 1124 which extends along the upper rim 134 back to a second end point 1130 at the interface 4. A second pathway 1115 extends in a first section 1121 from a first end point 1119 above the first end point 1118 of the first pathway 1114 and along the upper rim to a position above the users first ear, at which point it transitions into a second section 1123 which extends along the fifth region 109 across the top of the user's head to a position above the user's second ear. At this position in the pathway 1115 transitions to a third section 1125 which extends forwardly along the upper rim to a second end point 1131 opposite the first end point 1119 and above the second end point 1130 of the first pathway 1114. As with previous embodiments, the interface 4 incorporates a controller 140 into which tension lines feed from the first and second pathways 1114 and 1115.

It will be appreciated that the first sections 1120 and 1121 of the first and second pathways 1114 and 1115 respectively and the third sections 1124 and 1125 of the first and second pathways 1114 and 1115 respectively may be combined into single pathway portions along the upper rim 134 so that two tension lines are contained within each portion until the combined pathway portions branch off into separate paths across the rear of the head and across the top of the head.

Another embodiment that is based on the same positioning web shown in FIGS. 15A and 26 has a pathway path as shown in FIG. 30. In this embodiment, a first pathway 1214 (comprising sections 1220, 1222 and 1224) extends extend from the fifth region 109 on the first side, then around the fourth region 108 from the first side to the second side and then into the fifth region 109 on the second side. A second pathway 1215 (comprising sections 1221, 1223 and 1225) extends from the fifth region 109 on the first side to the third region 106 on the first side and then along the third region 106 from the first side to the second side and into the fifth region 109 on the second side

More specifically, the first section 1220 of the first pathway 1214 extends from a first end point 1218 along the fifth region 109 to a position above the user's first ear where it transitions into a second section 1222 which extends along the upper rim 134 and traverses the user's face to a position above the user's second ear including crossing the interface 4 at a location which coincides with the user's upper lip. At the position above the user's second ear, the second section 1222 transitions into a third section 1224 which extends up along the fifth region 109 to a second end point 1230 opposite the first end point 1218. The second pathway 1215 commences from a first end point 1219 adjacent the first end point 1218 of the first pathway 1214 and extends as the first section 1221 along the fifth region 109 to position above the user's first ear and then transitions to the second section 1223 which extends diagonally downwardly at the rear of the user's head to the lower rim 132 and then extends diagonally upwardly to a position above the user's second ear. From this point, the pathway 1215 transitions to a third section 1225 which extends along the fifth region 109 to a second end point 1231 which is opposite the first end point 1219 and adjacent the second end point 1230 of the first pathway 1214.

The controller 140 is located on the fifth region 109 between all of the end points 1218, 1230, 1219 and 1231 and performs the same function of adjusting the length of the tension lines 150 contained in the pathways 1214 and 1215.

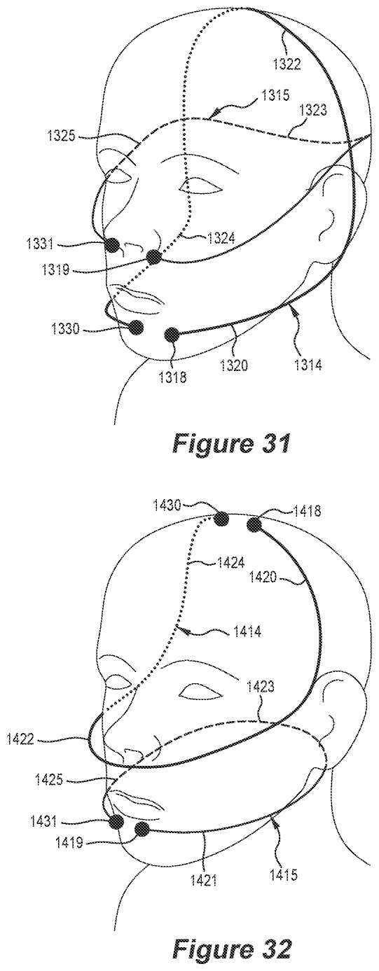

In a further embodiment shown in FIG. 31 which utilises the same positioning web in FIGS. 15A and 26, a first pathway 1314 (comprising sections 1320, 1322 and 1324) extends rearwardly along the first region 102 on the first side, then across the fifth region 109 to the second side and the forwardly along the first region 102. A second pathway 1315 (comprising sections 1321, 1323 and 1325) extends rearwardly along the fourth region 108 on the first side, along the third region 106 about the rear of the positioning web and then forwardly along the fourth region 108 on the second side

More specifically, the first pathway 1314 commences at a first end point 1319 at a lower portion of the interface 4 which coincides with the chin of the user and extends in a first section 1320 along the lower rim 132 to a position below the first ear of the user. The pathway 1314 then transitions into a second section 1322 which extends upwardly from the lower rim 132, behind the first ear and into fifth region 109 where the pathway 1315 continues across the fifth region 109 and descends to the lower rim 132 behind the second ear. The pathway 1314 then transitions into a third section 1324 which continues along the lower rim 132 toward the interface 4 and terminates at a second end point 1330 at a lower portion of the interface 4 which coincides with the chin of the user. The second pathway 1315 has a first end point 1319 which is located on an upper portion of the interface 4 to coincide with the upper lip of the user and extends in a first section 1321 along the upper rim 134 to a position above the first ear of user. The pathway 1315 then transitions to a second section 1322 which extends around the rear of the user's head and, in doing so, dips down toward the lower rim 132. The pathway 1315 then transitions into a third section 1325 which extends along the upper rim 134 above the second ear and continues along the upper rim 134 to the interface 4 and terminates at a second end point 1331, opposite to the first end point 1319, which coincides with the upper lip of the user. The length of tension line in the first and second pathways 1314 and 1315 is controlled by a controller that is associated with the interface 4 between the end points 1318, 1319, 1330 and 1331.

A variation of this embodiment is shown in FIG. 32. In this embodiment, a first pathway 1414 (comprising 1420, 1422 and 1425) extends rearwardly along the first region 102 on the first side, then along the second region 104 from the first side to the second side and then forwardly along first region 102 on the second side. A second pathway 1415 (comprising sections 1421, 1423 and 1425) extends from the fifth region 109 to the fourth region 108 on the first side, along the fourth region 108 from the first side to the second side and then into the fifth region on the second side

More specifically, the first section 1420 of the first pathway 1414 extends from a first end point 1418 along the fifth region 109 to a position above the users first ear and then transitions into a second section 1422 which extends along with the upper rim 134 by traversing the user's face from the first side to the second side. The first pathway 1414 crosses the interface 4 at a location that coincides with the user's upper lip and continues to a position above the user's second ear. At that point, the pathway 1414 transitions into a third section 1424 which extends upwardly into the fifth region 109 to the second end point 1430. A controller 140 is located on the fifth region 109 between the first and second end points 1418 and 1430 to control the length of tension line within the first pathway. The controller 140 is in the same form as described above in respect of the embodiment shown in FIGS. 14 to 16.

The second pathway 1415 starts and ends at the interface 4. In particular, it starts at end point 1419 which coincides with the user's chin and extends rearwardly along the first side along the lower rim 132, about the rear of the user's head and then forwardly along the lower rim 132 to an end point 1431. The length of a tension line located in the second pathway 1415 is adjusted by a controller 140 associated with the interface 4. Again, the controller 140 is in the same form as described above in respect of the embodiment shown in FIGS. 14 to 16.

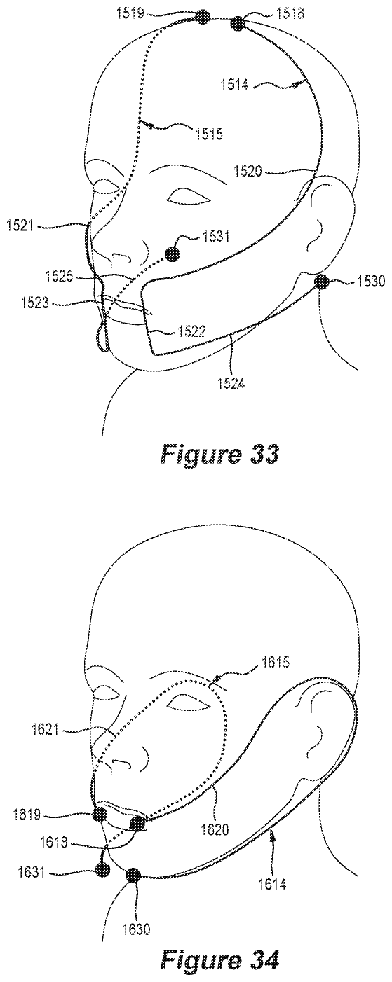

An alternative embodiment, having two separate pathways which don't intersect, is shown in FIG. 33. In this embodiment, a first pathway 1514 (comprising 1520, 1522 and 1524) extends from the fifth region 109 on the first side and then forwardly along the fourth region 108 before transitioning to the first region 102 and then extending rearwardly along the first region 102 on the first side. The second pathway 1515 (comprising 1521, 1523 and 1525) extends from the fifth region 109 on the second side and then forwardly along the fourth region 108 before transitioning to the first region 102 and extending rearwardly along the first region 102 on the second side.

More specifically, the first pathway 1514 has a first end point 1518 in the fifth region 109 and a first section 1520 extending along the fifth region 109 and into the upper rim 134 above a first ear of the user. The first section 1520 continues along the upper rim 134 toward the interface 4 and then transitions into a second section 1522 which extends downwardly from the upper rim 134 to the lower rim 132 without extending diagonally or laterally across the interface 4. At the lower rim 132, the pathway 1514 transitions into a third section 1524 that returns along the positioning web 112 along the lower rim 132 to a position below the first ear of the user or slightly further along the lower rim 132 toward the rear of the head. A second pathway 1515 has a path that is a mirror image of the first pathway 1514 in that it originates on the fifth region 109 at a first end point 1519 on an opposite side of a controller 140 to the first end point 1518 of the first pathway 1514 and extends away from the first pathway 1514 along on the opposite side of the fifth region 109 in a first section 1521. The first section 1521 extends into the upper rim 134 above a second ear of the user. The first section 1521 then continues along the upper rim 134 toward the interface 4 and then transitions into a second section 1523 which extends downwardly from the upper rim 134 to the lower rim 132 without extending diagonally or laterally across the interface 4. At the lower rim 132, the pathway 1515 transitions into a third section 1525 that returns along the positioning web 112 along the lower rim 132 of the headgear 110 to a position below the second ear of the user or slightly further along the lower rim toward the rear of the head.

A controller 140, in the same form as the controller 140 described above in respect of the embodiment shown in FIGS. 14 to 16, is located on the fifth region 109 between all of the first end points 1518 and 1519 for adjusting the length of tension lines located in the respective pathways 1514 and 1515. Ends of tension lines 150 remote from the controller 140 exit the pathways 1514 and 1515 at the second end points 1530 and 1531 and are anchored to the positioning web at that point. Accordingly, tension in the tension lines 150 is transferred to the positioning web 112 at the anchor point and is distributed through the positioning web 112 to counteract the tension.

Another alternative pathway path is shown in the embodiment illustrated in FIG. 34. In that embodiment, a first pathway 1614 (comprising sections 1620, 1622 and 1624) extends rearwardly along the fourth region 108 on the first side and then forwardly along the first region 102 on the first side. The second pathway 1615 (comprising sections 1621, 1623 and 1625) extends rearwardly along the fourth region 108 on the second side and then forwardly along the first region 102 on the second side.

The first pathway portion may extend rearwardly along the first region on the first side, then forwardly along the fourth region on the first side and then transition into the first region at or adjacent to a respiratory interface region and the second pathway portion may extend rearwardly along the first region on the second side and then forwardly along the fourth region on the second side and then transition into the first region at or adjacent to a respiratory interface region.

More specifically, the first section 1620 of the first pathway 1614 extends from a first end point 1618 at an upper portion of the interface 4 and extends along the upper rim 134 to a position slightly behind the first ear of the user. At this point the pathway 1614 then transitions from the upper rim 132 to the lower rim 134 about the rear of the user's first ear. The pathway 1614 then returns along the lower rim 132 back toward the interface 4 where the pathway 1614 terminates in a second end point 1630 at a lower portion of the interface 4 below the first end point 1618. The second pathway 1615 is a mirror image of the first pathway 1614 in that it originates at a first end point 1619 at an upper portion of the interface 4 and extends along the upper rim 134 to a position slightly behind the second ear of the user. At this point the pathway 1615 then transitions from the upper rim 132 to the lower rim 134 about the rear of the user's first ear. The second pathway 1615 then returns along the lower rim 132 back toward the interface 4 where the pathway 1615 terminates in a second end point 1631 at a lower portion of the interface 4 below the first end point 1619 and opposite to the first end point 1618 of the first pathway 1614.

A controller 140 is linked to tension lines located within the first and second pathways 1614 and 1615 to adjust the length of the tension lines contained within the pathways 1614 and 1615. Both ends of each tension line may be linked to the controller 140. Alternatively, one end of each tension line may be linked to the controller 140. For example, the ends of the tension lines which enter the first end points 1618 and 1619 may be linked with the controller 140 and the other ends of the tension lines which enter the second end points 1630 and 1631 may be anchored to the interface 4 or anchored to the positioning web 112. In an alternate example, the ends of the tension lines which enter the second end points 1630 and 1631 may be linked with the controller 140 and the other ends of the tension lines which enter the first end points 1618 and 1619 may be anchored to the interface 4 or anchored to the positioning web. As another example, diagonally opposed ends of the tension lines may be linked with the controller 140 and the other ends may be anchored to the interface 4 or anchored to the positioning web 112.

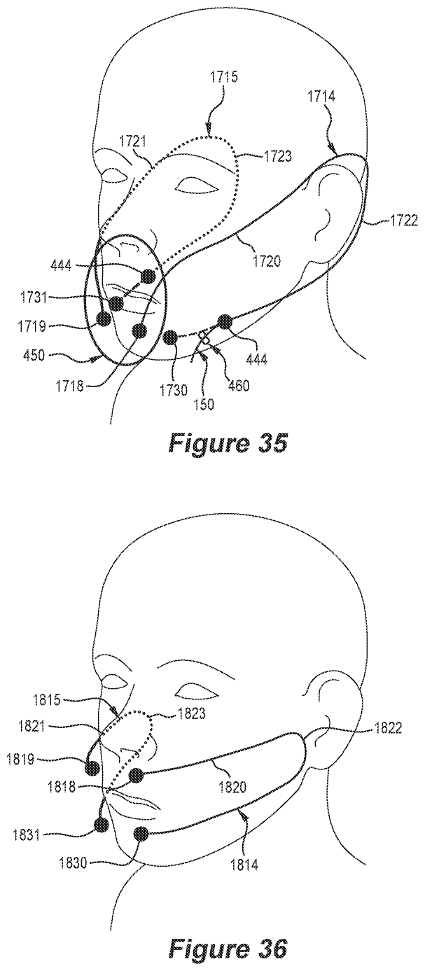

A variation of the embodiment shown in FIG. 34 is shown in FIG. 35. In this embodiment, a first pathway 1714 (comprising sections 1720 and 1722) extends from the first region 102 to the fourth region 108 at or adjacent to a respiratory interface region 450 and then extends rearwardly along the fourth region 108 on the first side, then forwardly along the first region 102 on the first side. The second pathway 1715 (comprising sections 1721 and 1723) extends from the first region 102 to the fourth region 108 at or adjacent to the respiratory interface region 450 on the second side and then extends rearwardly along the fourth region 108 on the second side and then forwardly along the first region 102 on the second side.

More specifically, first end points 1718 and 1719 of the first and second pathways 1714 and 1715 respectively are located at a lower portion of the interface 4, which coincides with a user's chin. Therefore, the first sections 1720 and 1721 of the respective pathways 1714 and 1715 extend upwardly from the lower portion of the interface 4 to the upper rim 134 and then extend along the upper rim 134 to a position slightly behind the first and second ears, respectively, of the user. At this point the pathways 1714 and 1715 transition from the first sections 1720 and 1721 into second sections 1722 and 1723 which transition from upper rim 132 to the lower rim 134 about the rear of the user's ears and then return along the lower rim 132 back toward the interface 4 where the pathways 1714 and 1715 terminate in respective second end points 1730 and 1731 at the lower portion of the interface 4, adjacent the first end points 1718 and 1719.

The tension lines 150 incorporate break-fit connections as described above in respect of the embodiment shown in FIG. 19. That is, the ends of the tension lines which enter the second ends 1730 and 1731 have a tab 460 which is releasably fastenable to an anchor point on the interface 4 or a point on the positioning web 112. Releasing the tab 460 from the anchor point releases the tension in the tension line to allow doffing of the headgear. Conversely, when donning the headgear, fastening the tab 460 to the anchor point returns the same tension to the tension lines 150 before they were released. This means that the headgear can be donned (including fitting) without adjustment of the tension lines 150 via the controller 140.

Another embodiment shown in FIG. 36 is a variation of the embodiment shown in FIG. 33. In this embodiment, a first pathway 1814 (comprising sections 1820 and 1822) extends rearwardly along the fourth region 108 on the first side and then forwardly along the first region 102 on the first side. A second pathway 1815 (comprising sections 1821 and 1823) extends rearwardly along the fourth region 108 on the second side and then forwardly along the first region 102 on the second side.

More specifically, a first section 1820 of the first pathway 1814 extends from a first end point 1818 at an upper portion of the interface 4 and extends along the upper rim 134 to a position slightly in front of the first ear of the user. At this point the pathway 1814 path has a curved arc shape 1822 which transitions from the upper rim 134 to the lower rim 132 in front of the user's ear and then returns along the lower rim 132 back toward the interface 4 where the pathway 1814 terminates in a second end point 1830 at a lower portion of the mask below the first end point 1818. The second pathway 1815 is a mirror image of the first pathway 1814 in that it originates at a first end point 1819 at an upper portion of the interface 4 laterally opposite to the first end point 1818 and extends along the upper rim 134 to a position in front of the second ear of the user. At this point the pathway 1815 has a curved arc shape 1823 which transitions from the upper rim 134 to the lower rim 132 in front of the user's second ear and then returns along the lower rim 132 back toward the interface 4 where the pathway 1815 terminates in a second end point 1831 at a lower portion of the interface 4 below the first end point 1819 and opposite to the first end point 1818 of the first pathway 1814.

As with the embodiment in FIG. 34, a controller 140 is associated with the interface 4 and is linked to tension lines located within the first and second pathways 1814 and 1815 to adjust the length of the tension lines located within the pathways 1814 and 1815. Both ends of each tension line may be linked to the controller 140. Alternatively, one end of each tension line may be linked to the controller 140. For example, the ends of the tension lines which enter the first end points 1818 and 1819 may be linked with the controller 140 and the other ends of the tension lines which enter the second end points 1830 and 1831 may be anchored to the interface 4 or anchored to the positioning web 112. In an alternate example, the ends of the tension lines which enter the second end points 1830 and 1831 may be linked with the controller 140 and the other ends of the tension lines which enter the first end points 1818 and 1819 may be anchored to the interface 4 or anchored to the positioning web 112. As another example, diagonally opposed ends of the tension lines may be linked with the controller 140 and the other ends may be anchored to the interface 4 or anchored to the positioning web 112.

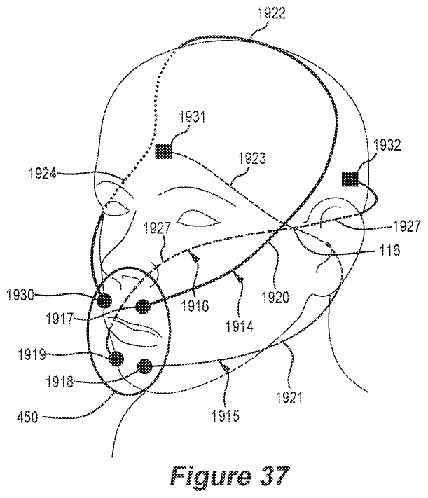

A further embodiment is shown in FIG. 37 and comprises a first pathway 1914, second pathway 1915 and third pathway 1916. The first pathway 1914 is the same as the second pathway 1115 of the embodiment shown in FIG. 29. That is, the first pathway 1914 extends in a first section 1920 from a first end point 1917 which is at or adjacent to respiratory interface region 450. The first pathway 1914 extends along the upper rim 134 to a position above the user's first ear. The first pathway 1914 transitions into a second section 1922 which extends along the fifth region 109 across the top of the user's head to a position above the user's second ear. At this position, the pathway 1914 transitions to a third section 1924 which extends forwardly along the upper rim 134 to a second end point 1930 opposite the first end point 1917.

The second pathway 1915 commences at a first end point 1918 which is at or adjacent the respiratory interface region 450. The first end point 1918, as shown in the FIG. 37, is located below the first end point 1917 of the first pathway 1914. The second pathway 1915 extends in a first section 1921 along the lower rim 132 to a position below the user's first ear. The second pathway 1915 then transitions into a second section 1923 which extends around the rear of the head from below the user's first ear to above the user's second ear. The second pathway 1915 terminates at a second end point 1931 above the user's second ear. The third pathway 1916 is a mirror image of the second pathway 1915 in that it originates at a first end point 1919 at a lower part of the interface region below the second end point 1930 of the first pathway 1914. The third pathway 1916 then extends in a first section 1925 along the lower rim 132 to a position below the user's second ear. The second pathway 1916 then transitions into a second section 1927 which extends around the rear of the head from below the user's second ear to above the user's first ear. The third pathway 1916 terminates at a second end point 1932 above the user's second ear. The first and second pathways 1915 and 1916 cross-over at an intersection point 116. The intersection point 116, as shown in FIG. 37, is at the central rear of the positioning web 112. Furthermore, it may be equally spaced from the upper and lower rims 134 and 132. Alternatively, it may be located at other positions. For instances, the intersection point 116 may be located closer to the upper rim 143 or closer to the lower rim 132.

As with previous embodiments, a controller 140 may be disposed in the respiratory interface region 450 to control the length of tension line 150 deployed in each of the first, second and third pathways 1914, 1915 and 1916 feed into the controller 140. More specifically, both ends of a tension line 150 disposed in the first pathway 1914 feeds into the controller 140 and one end of each tension line 150 disposed in the second and third pathways 1915 and 1916 feeds into the controller 140. Ends of tension lines 150 remote from the controller 140 exit the second and third pathways 1915 and 1916 at the respective second end points 1931 and 1932 and are anchored to the positioning web 112 at or adjacent to the second end points 1931 and 1932. Accordingly, tension in the tension lines 150 is transferred to the positioning web 112 at the anchor point and is distributed through the positioning web 112 to counteract the tension.

The anchoring of the tension line 150 to the positioning web 112 may be permanent or may enable release, and optionally re-anchoring, of the tension line. In either case, the positioning web 112 and the tension line 150 may include co-operable formations that enable anchoring of the tension line 150 to the positioning web 112. The anchoring may include fixing the tension line to itself to form a loop which captures a portion of the positioning web 112 in the loop.

Those skilled in the art of the present invention will appreciate that many variations and modifications may be made to the preferred embodiment without departing from the spirit and scope of the present invention.

In the claims which follow, and in the preceding description, except where the context requires otherwise due to express language or necessary implication, the word “comprise” and variations such as “comprises” or “comprising” are used in an inclusive sense, i.e. to specify the presence of the stated features but not to preclude the presence or addition of further features in various embodiments of the apparatus and method as disclosed herein.

In the foregoing description of preferred embodiments, specific terminology has been resorted to for the sake of clarity. However, the invention is not intended to be limited to the specific terms so selected, and it is to be understood that each specific term includes all technical equivalents which operate in a similar manner to accomplish a similar technical purpose. Terms such as “front” and “rear”, “inner” and “outer”, “above”, “below”, “upper” and “lower” and the like are used as words of convenience to provide reference points and are not to be construed as limiting terms. These terms and the terms “vertical” and “horizontal” when used in reference to the headgear throughout the specification, including the claims, refer to orientations relative to the normal operating orientation of the headgear when fitted to a user's head in an upright position.

Furthermore, the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, for example, aspects of one embodiment may be combined with aspects of another embodiment to realize yet other embodiments. Further, each independent feature or component of any given assembly may constitute an additional embodiment.

1. A headgear for a respiratory interface, the headgear comprising a positioning web having a height and a length that extends at least partly about a head of a user when the headgear is fitted to the user and the positioning web including:
at least one pathway for at least one tension line, the at least one pathway comprising;
a first pathway portion extending along at least part of the length of the positioning web, and
a second pathway portion extending along at least part of the length of the positioning web, wherein the first pathway portion and second pathway portion are spaced apart by a varying distance as they extend along the positioning web;
and wherein the positioning web:
maintains a positioning of the at least one pathways about the head of the user; and
is expandable and contractible between an operative configuration when fitted to the user and an expanded configuration to facilitate donning and doffing of the headgear respectively.

2. The headgear of claim 1, wherein the first pathway portion and the second pathway portion are portions of a single pathway.

3. The headgear of claim 2, wherein the positioning web comprises a first pathway and a second pathway, wherein the first pathway comprises the first pathway portion and the second pathway comprises the second pathway portion.

4. The headgear of claim 2, wherein the first pathway portion and the second pathway portion intersects.

5. The headgear of claim 4, wherein the first pathway portion and the second pathway portion may intersect at least once at a point where a distance between the first pathway portion and the second pathway portion is zero.

6. The headgear of claim 1, wherein the positioning web is elastic or inelastic.

7. The headgear of claim 6, wherein an elasticity of the positioning web is anisotropic.

8. The headgear of claim 1, wherein the headgear further comprises one or more tension lines respectively disposed in the at least one pathway.

9. The headgear of claim 8, wherein each of the one or more tension lines is operable to hold the positioning web in the operative configuration when the headgear has been donned.

10. The headgear of claim 8, wherein the one or more tension lines comprise a first tension line portion and a second tension line portion and wherein the first tension line portion co-operates with the first pathway portion to extend along the positioning web, and the second tension line portion co-operates with the second pathway portion to extend along the positioning web.

11. The headgear of claim 8, wherein the one or more tension lines are less capable of expanding and contracting compared to an expandability and contractibility of the positioning web.

12. The headgear of claim 8, wherein the one or more tension lines are less elastic than the positioning web.

13. The headgear of claim 8, wherein the one or more tension lines may be sufficiently elastic to stretch to account for small changes in tension.

14. The headgear of claim 8, wherein the one or more tension lines is substantially inelastic at tensions associated with blow-off and tube-pull forces.

15. The headgear of claim 8, wherein one of the one or more tension lines is inelastic compared to an expandability and contractibility of the positioning web for tensions of at least double a blow-off and tube-pull forces.
